(19) United States
(12) Patent Application Publication
(10) Pub. No.: US 2024/0287108 A1
(43) Pub. Date: Aug. 29, 2024

(54) nSMASE2 INHIBITOR PRODRUGS WITH ENHANCED ORAL AND BRAIN EXPOSURES

(71) Applicants: Rana RAIS, Baltimore, MD (US); The Johns Hopkins University, Baltimore, MD (US)

(72) Inventors: Rana RAIS, West Friendship, MD (US); Barbara SLUSHER, Kingsville, MD (US); Arindom S. PAL, Baltimore, MD (US)

(21) Appl. No.: 18/566,922

(22) PCT Filed: Jun. 7, 2022
(86) PCT No.: PCT/US2022/032538

§ 371 Date: Dec. 4, 2023

Related U.S. Application Data
(60) Provisional application No. 63/197,691, filed on Jun. 7, 2021.

(51) Int. Cl. C07F 9/6558; A61K 31/4178; A61K 31/439; A61K 31/4439; A61K 31/454; A61K 31/4545; A61K 31/675; C07D 409/04; C07D 409/14; C07D 471/08

(52) U.S. Cl. CPC C07F 9/65586; A61K 31/4178; A61K 31/439; A61K 31/4439; A61K 31/454; A61K 31/4545; A61K 31/675; C07D 409/04; C07D 409/14; C07D 471/08

(57) ABSTRACT

Prodrugs of small molecule inhibitors of neutral sphingomyelinase 2 (nSMase2) and their use for treating neurodegenerative diseases, such as, neurodegenerative diseases associated with high levels of ceramide, including, but not limited to Alzheimer's disease (AD), HIV-associated neurocognitive disorder (HAND), multiple sclerosis (MS), and amyotrophic lateral sclerosis (ALS), and, in other aspects, for treating cancer, are provided.

### FEDERALLY SPONSORED RESEARCH OR DEVELOPMENT

This invention was made with United States Government support under AG059799 and AG063831 awarded by the National Institutes of Health. The U.S. Government has certain rights in the invention.

### BACKGROUND

Exosomes are small extracellular vehicles (EVs) carrying protein and RNA that are shed from cells in response to various stimuli. Cumulative evidence suggests that under disease conditions, EVs can carry pathological cargo and play an active role in disease propagation. The hydrolysis of sphingomyelin (SM) to ceramide by nSMase2 is a major regulator in several independent routes of biogenesis and release of EVs. Trajkovic et al., 2008. While transient increases in nSMase2 activity are part of normal physiological function, chronically activated nSMase2 and subsequent EV secretion have been implicated in modulating the immune response to brain inflammation, Dickens et al., cancer metastasis, Kosaka et al., 2013, amyloid deposition, Dinkins et al., 2016; Dinkins et al., 2014; Sardar Sinha et al., 2018, Tau protein propagation, Asai et al., 2015, and HIV infection. Barclay et al., 2017: Dalvi et al., 2017; Hu et al., 2021: Sun et al., 2017. Moreover, genetic and pharmacological inhibition of nSMase2 have been demonstrated to reduce EV secretion.

### SUMMARY

In some aspects, the presently disclosed subject matter provides a compound of formula (I):

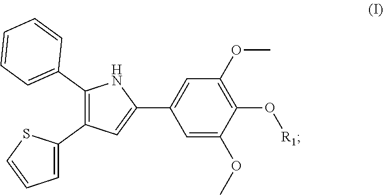

wherein:

R₁ is —C(═O)—R₂ or —(CH₂)ₙ—O—P(═O)(OH)(OR₃), wherein:

n is an integer selected from the group consisting of 0, 1, 2, 3, and 4;

R₂ is selected from the group consisting of substituted or unsubstituted C₁-C₈ straight-chain or branched alkyl, —NR₄R₅, substituted or unsubstituted cycloalkyl or cycloheteroalkyl, substituted or unsubstituted aryl or heteroaryl, substituted or unsubstituted bicycloalkyl or bicycloheteroalkyl, —O—CH(R₆)—O—(C═O)—R₇, and —CH(R₈)(NR₉)—(C═O)—CH(NR₁₀R₁₁)—R₁₂;

wherein:

R₄, R₅, R₈, R₉, R₁₀, and R₁₁ are each independently selected from the group consisting of H and substituted or unsubstituted straight-chain or branched C₁-C₈ alkyl:

R₆ is selected from the group consisting of substituted or unsubstituted straight-chain or branched C₁-C₄ alkyl and substituted or unsubstituted aryl or heteroaryl; and

R₇ and R₁₂ are each independently substituted or unsubstituted straight-chain or branched C₁-C₄ alkyl,

R₃ is selected from the group consisting of H and —(CH₂)ₘ—O—C(═O)—O—R₁₃, wherein m is an integer selected from the group consisting of 1, 2, 3, and 4, and R₁₃ is substituted or unsubstituted C₁-C₄ alkyl;

and pharmaceutically acceptable salts thereof.

In particular aspects, R₁ is —C(═O)—R₂. In certain aspects, R₂ is substituted or unsubstituted C1-C₈ straight-chain or branched alkyl. In more certain aspects, R₂ is selected from the group consisting of methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, and n-octyl.

In particular aspects, R₂ is —NR₄R₅.

In more particular aspects, R₂ is selected from the group consisting of

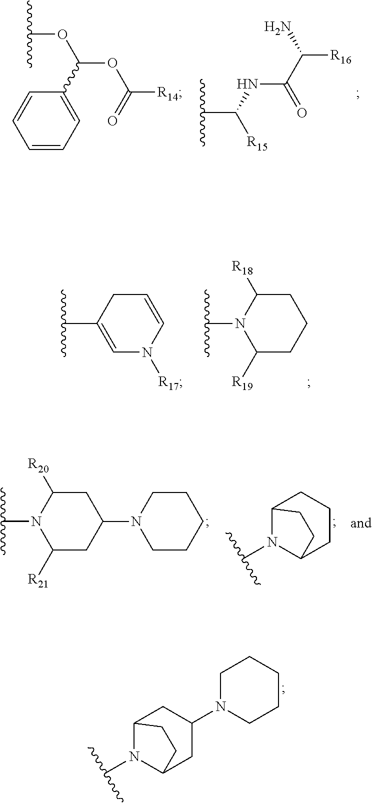

wherein R₁₄, R₁₅, R₁₆, R₁₈, and R₁₉ are each independently selected from the group consisting of substituted or unsubstituted straight-chain or branched alkyl, and R₁₇, R_{20,} and R₂₁ are each independently selected from the group consisting of H or C₁-C₄ substituted or unsubstituted C₁-C₄ straight-chain or branched alkyl.

In some aspects, R₁ is —(CH₂)ₙ—O—P(═O)(OH)(OR₃). In certain aspects, n is 0 or 1. In particular aspects, R₃ is H or —CH₂—O—C(═O)—O—R₁₃, wherein R₁₃ is selected from the group consisting of methyl, ethyl, n-propyl, isopropyl, n-butyl, isobutyl, sec-butyl, and tert-butyl.

In yet more particular aspects, the compound of formula (I) is selected from the group consisting of:

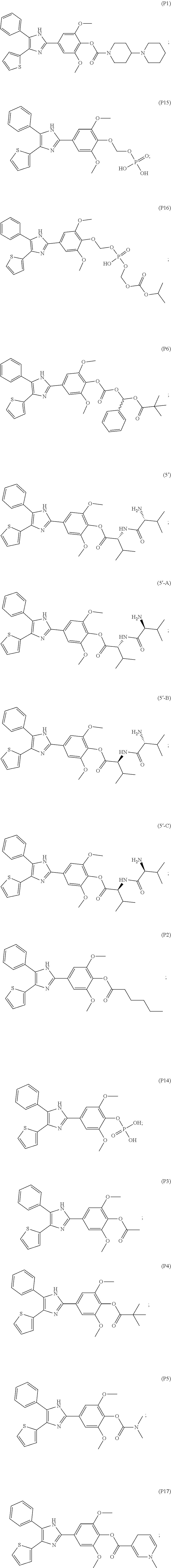

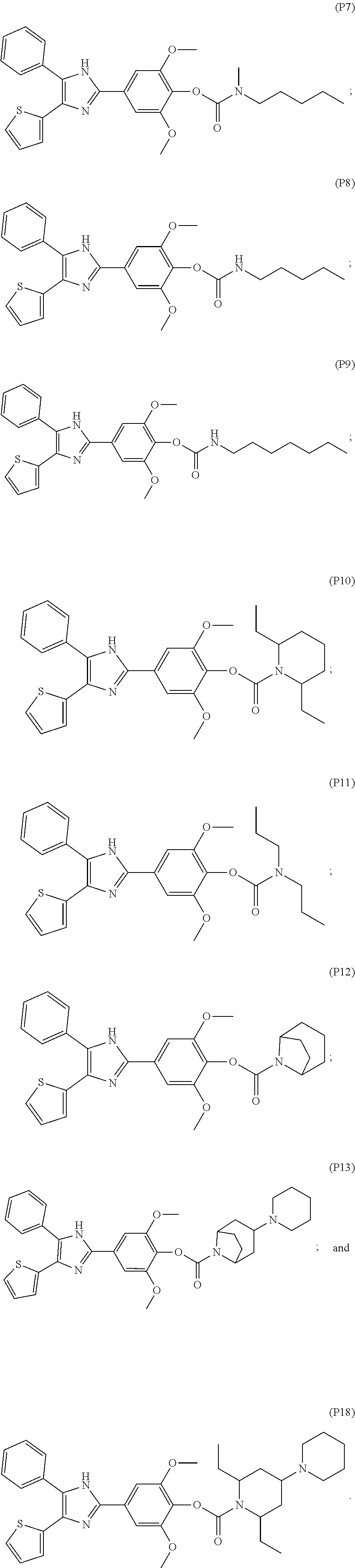

In some aspects, the presently disclosed subject matter provides a pharmaceutical formulation comprising a compound of formula (I) and a pharmaceutically acceptable carrier. In certain aspects, the pharmaceutical formulation comprises one or more of a polyvinyl caprolactam-polyvinyl acetate-polyethylene glycol graft copolymer (PCL-PVAc-PEG), a caprylocaproyl macrogol-8 glyceride, and a cyclodextrin.

In other aspects, the presently disclosed subject matter provides a method for treating a condition, disease, or disorder associated with an increased neutral sphingomyelinase 2 (nSMase2) activity or expression, the method comprising administering to a subject in need of treatment thereof an effective amount of a compound of formula (I) and pharmaceutically acceptable salts thereof.

In particular aspects, the condition, disease, or disorder is associated with an elevated level of ceramide in the subject in need of treatment compared to a control subject not afflicted with the condition, disease, or disorder.

In certain aspects, the administration of an effective amount of a compound of formula (I) to the subject decreases the (nSMase2) activity or expression or decreases a level of ceramide in the subject.

In some aspects, the condition, disease, or disorder is associated with an extracellular vesicle-mediated condition, disease, or disorder.

In certain aspects, the extracellular vesicle-mediated disease is selected from the group consisting of a neurological, an oncological, an inflammatory, and an infectious condition, disease, or disorder.

In particular aspects, the neurological condition, disease, or disorder is selected from the group consisting of Alzheimer's disease (AD), Parkinson's disease, HIV-associated neurocognitive disorder (HAND), multiple sclerosis (MS), amyotrophic lateral sclerosis (ALS), frontotemporal dementia (FTD), prion diseases, Duchenne muscular dystrophy (DMD), and retinal degeneration.

In particular aspects, the oncological condition, disease, or disorder is selected from the group consisting of breast cancer, cervical cancer, colon cancer, colorectal cancer (CRC), duodenal cancer, gastric cancer, lung cancer, multiple myeloma, oral cancer, pancreatic cancer, prostate cancer, skin cancer, and a cancer therapy in combination with immunotherapy to enhance systemic antitumor immunity.

In particular aspects, the inflammatory condition, disease, or disorder is selected from the group consisting of an inflammatory airway disease, including an allergic airway inflammation, an ischemia-reperfusion injury, including cerebral ischemia and hepatic ischemia-reperfusion injury, sepsis, atherosclerosis, myocardial infarction, and inflammatory bowel disease.

In particular aspects, the infectious condition, disease, or disorder comprises a viral infection selected from the group consisting of HIV, Zika virus, rabies virus, Dengue virus, hepatitis C (HCV), hepatitis E (HEV), cytomegalovirus (HCMV), Newcastle disease virus (NDV), and Langat virus.

In particular aspects, the infectious disease is related to a toxin produced from a bacterial infection, including Shiga toxin released by Escherichia coli, and epsilon toxin, released by Clostridum perfringens.

In other aspects, the presently disclosed subject matter provides a method for inhibiting neutral sphingomyelinase 2 (nSMase2), the method comprising administering to a subject, cell, or tissue an amount of a compound of formula (I) effective to inhibit nSMase2.

In yet other aspects, the presently disclosed subject matter provides for the use of a compound of formula (I) for preparing a medicament for treating a condition, disease, or disorder associated with an increased neutral sphingomyelinase 2 (nSMase2) activity or expression.

Certain aspects of the presently disclosed subject matter having been stated hereinabove, which are addressed in whole or in part by the presently disclosed subject matter, other aspects will become evident as the description proceeds when taken in connection with the accompanying Examples and Drawings as best described herein below.

### BRIEF DESCRIPTION OF THE DRAWINGS

Having thus described the presently disclosed subject matter in general terms, reference will now be made to the accompanying Figures, which are not necessarily drawn to scale, and wherein:

FIG. 1A, FIG. 1B, and FIG. 1C show single time-point pharmacokinetic screening of all prodrugs (P1-P18) and DPTIP in mice dosed orally at 10 mg/kg DPTIP equivalent. (FIG. 1A) Plasma levels of released DPTIP from the prodrugs, (FIG. 1B) Brain levels of released DPTIP from the prodrugs, and (FIG. 1C) Brain to plasma ratio of released DPTIP at 2 hours;

FIG. 2A, FIG. 2B, FIG. 2C, and FIG. 2D show time-dependent in vivo PK analysis of DPTIP (FIG. 2A) P1 (FIG. 2B) P18 (FIG. 2C) and their PK parameters (FIG. 2D) in mice. DPTIP, P1, and P18 were dosed orally at 10 mg/kg DPTIP equivalent dose. Data expressed as mean±SEM, n=3.

FIG. 3A. FIG. 3B, and FIG. 3C demonstrate the effects of orally administered P18 in a mouse model of acute brain injury. Mice were pretreated 30 min prior to intra-striatal injections of saline or IL-1β (0 h) with either vehicle, or P18 (3 and 10 mg/kg). Four hours later, mice were sacrificed and the astrocyte-derived EVs released into plasma, striatal nSMase2 activity, and plasma and brain levels of released DPTIP were quantified. (FIG. 3A) GFP labeled EVs in plasma under different treatments. ###p<0.001 (compared to saline+vehicle group); ***p<0.001 (compared to IL1-β+vehicle group). Statistical analysis was done using a one-way ANOVA with Tukey's post hoc test, (FIG. 3B) nSMase2 activity in mouse striata under different treatments following IL-1β injection. n=4/group, bars represent mean±SEM. ####p<0.0001 (compared to saline+vehicle group); ****p<0.0001, ***p<0.001 and *p<0.05 (compared to IL-1β+vehicle group). Statistical analysis was done using a one-way ANOVA with Tukey's post hoc test. (FIG. 3C) Plasma and brain levels of released DPTIP in the same samples;

FIG. 4A and FIG. 4B show the oral pharmacokinetic screening of compounds P1-P18 and DPTIP in mice dosed at 10 mg/kg DPTIP equivalent. (FIG. 4A) Plasma levels of intact prodrugs, (FIG. 4B) Brain levels of intact prodrugs;

FIG. 5A, FIG. SB. and FIG. 5C show time-dependent in vivo pharmacokinetic analysis of intact Compound P1 (FIG. 5A), P6 (FIG. 5B) and P18 (FIG. 5C) and their detail pharmacokinetic parameters (FIG. 5D) in mice. Compounds were dosed orally at 10 mg/kg DPTIP equivalent. Data expressed as mean±SEM, n=3;

FIG. 6 shows the dog plasma pharmacokinetics of DPTIP and prodrugs P1 and P2; and

FIG. 7 shows the DPTIP release in formulations of prodrug P18 in 5% DMSO, 10% Tween-80 and 85% PBS (left panel) compared to formulations of prodrug P18 in Soluplus® (middle panel) and Labrasol® (right panel).

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. nSMASE2 Inhibitor Prodrugs with Enhanced Oral and Brain Exposures

In some embodiments, the presently disclosed subject matter provides prodrugs of small molecule inhibitors of neutral sphingomyelinase 2 (nSMase2) for the treatment of neurodegenerative diseases, such as, neurodegenerative diseases associated with high levels of ceramide, including, but not limited to, Alzheimer's disease (AD), multiple sclerosis (MS), amyotrophic lateral sclerosis (ALS) and HIV-associated neurocognitive disorders (HAND). The presently disclosed nSMase2 inhibitor prodrugs also could be used for the treatment of cancer.

### A. Representative Compounds of Formula (I)

In some embodiments, the presently disclosed subject matter provides a compound of formula (I):

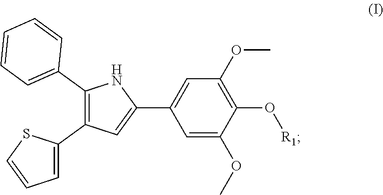

wherein:

R₁ is —C(═O)—R₂ or —(CH₂)ₙ—O—P(═O)(OH)(OR₃), wherein:

n is an integer selected from the group consisting of 0, 1, 2, 3, and 4;

R₂ is selected from the group consisting of substituted or unsubstituted C₁-C₈ straight-chain or branched alkyl, —NR₄R₅, substituted or unsubstituted cycloalkyl or cycloheteroalkyl, substituted or unsubstituted aryl or heteroaryl, substituted or unsubstituted bicycloalkyl or bicycloheteroalkyl, —O—CH(R₆)—O—(C═O)—R₇, and —CH(R₈)(NR₉)—(C═O)—CH(NR₁₀R₁₁)—R₁₂;

wherein:

R₄, R₅, R₈, R₉, R₁₀, and R₁₁ are each independently selected from the group consisting of H and substituted or unsubstituted straight-chain or branched C₁-C₈ alkyl;

R₆ is selected from the group consisting of substituted or unsubstituted straight-chain or branched C₁-C₄ alkyl and substituted or unsubstituted aryl or heteroaryl; and

R₇ and R₁₂ are each independently substituted or unsubstituted straight-chain or branched C₁-C₄ alkyl,

R₃ is selected from the group consisting of H and —(CH₂)ₘ—O—C(═O)—O—R₁₃, wherein m is an integer selected from the group consisting of 1, 2, 3, and 4, and R₁₃ is substituted or unsubstituted C₁-C₄ alkyl;

and pharmaceutically acceptable salts thereof.

In particular embodiments, R₁ is —C(═O)—R₂. In certain embodiments, R₂ is substituted or unsubstituted C₁-C₈ straight-chain or branched alkyl. In more certain embodiments, R₂ is selected from the group consisting of methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, and n-octyl.

In particular embodiments, R₂ is —NR₄R₅.

In more particular embodiments, R₂ is selected from the group consisting of

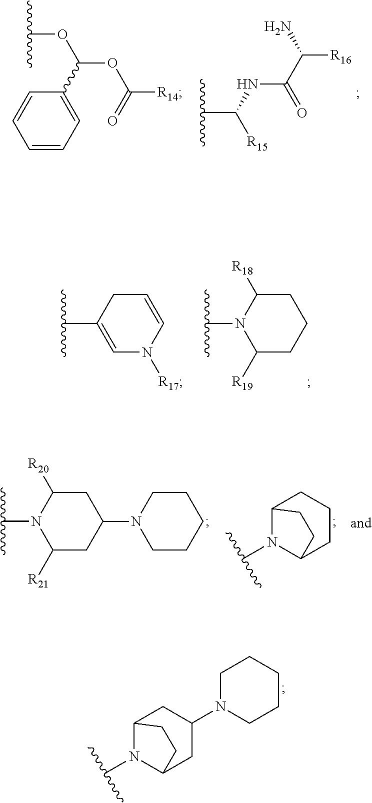

wherein R₁₄, R₁₅, R₁₆, R₁₈, and R₁₉ are each independently selected from the group consisting of substituted or unsubstituted straight-chain or branched alkyl, and R₁₇, R₂₀, and R₂₁ are each independently selected from the group consisting of H or C₁-C₄ substituted or unsubstituted C₁-C₄ straight-chain or branched alkyl.

In some embodiments, R₁ is —(CH₂)ₙ—O—P(═O)(OH)(OR₃). In certain embodiments, n is 0 or 1. In particular embodiments, R₃ is H or —CH₂—O—C(═O)—O—R₁₃, wherein R₁₃ is selected from the group consisting of methyl, ethyl, n-propyl, isopropyl, n-butyl, isobutyl, sec-butyl, and tert-butyl.

In yet more particular embodiments, the compound of formula (I) is selected from the group consisting of:

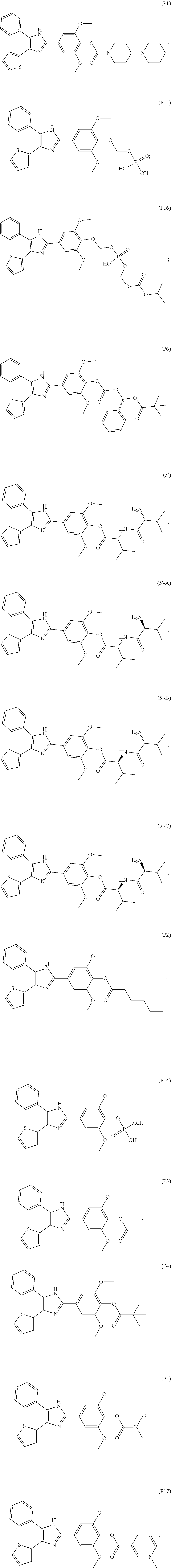

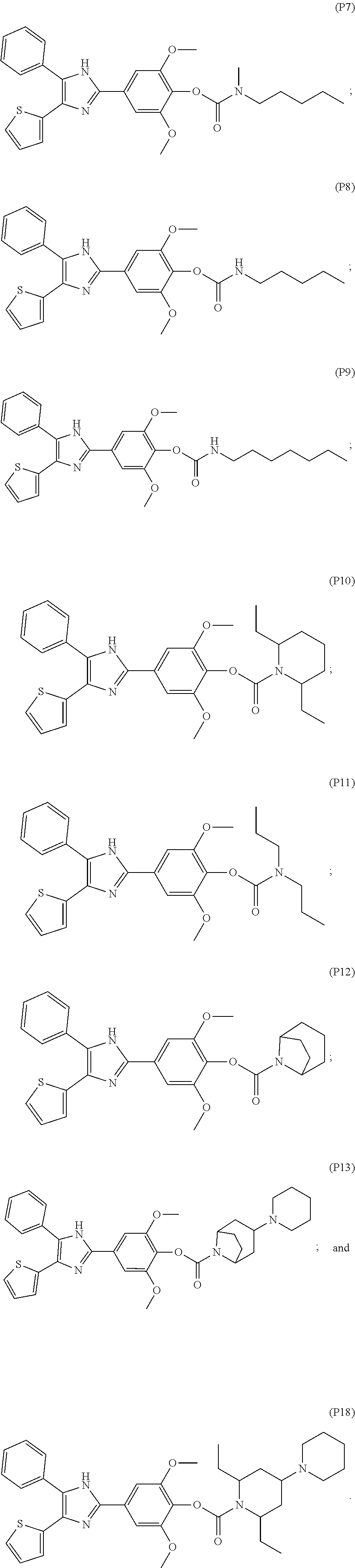

In some embodiments, the presently disclosed subject matter provides a pharmaceutical formulation comprising a compound of formula (I) and a pharmaceutically acceptable carrier. In certain embodiments, the pharmaceutical formulation comprises one or more of a polyvinyl caprolactam-polyvinyl acetate-polyethylene glycol graft copolymer (PCL-PVAc-PEG), a caprylocaproyl macrogol-8 glyceride, and a cyclodextrin.

B. Methods for Treating a Condition, Disease, or Disorder Associated with an Increased Neutral Sphingomyelinase 2 (Nsmase2) Activity or Expression

The presently disclosed subject matter provides a method for treating a condition, disease, or disorder associated with an increased neutral sphingomyelinase 2 (Nsmase2) activity or expression, the method comprising administering a compound of formula (I) to a subject in need of treatment thereof.

In some embodiments, the condition, disease, or disorder is associated with an elevated level of ceramide in the subject in need of treatment compared to a control subject not afflicted with the condition, disease, or disorder.

In particular embodiments, the administration of an effective amount of a compound of formula (I) to the subject decreases the (nSMase2) activity or expression or decreases a level of ceramide in the subject.

In some embodiments, the condition, disease, or disorder is associated with an extracellular vesicle-mediated condition, disease, or disorder. Extracellular vesicle-mediated conditions, diseases, or disorder include, but are not limited to, neurological, oncological, inflammatory, and infectious conditions, diseases, or disorders. See, for example, Tallon et al., 2021.

In some embodiments, the condition, disease, or disorder comprises a neurodegenerative condition, disease, or disorder. In particular embodiments, the neurodegenerative condition, disease, or disorder is selected from the group consisting of Alzheimer's disease (AD), Parkinson's disease, HIV-associated neurocognitive disorder (HAND), multiple sclerosis (MS), amyotrophic lateral sclerosis (ALS), frontotemporal dementia (FTD), prion diseases, Duchenne muscular dystrophy (DMD), and retinal degeneration.

In some embodiments, the condition, disease, or disorder is an oncological condition, disease, or disorder, including a cancer. In particular embodiments, the cancer is selected from the group consisting of breast cancer, cervical cancer, colon cancer, colorectal cancer (CRC), duodenal cancer, gastric cancer, lung cancer, multiple myeloma, oral cancer, pancreatic cancer, prostate cancer, skin cancer, and in combination with immunotherapy to enhance systemic antitumor immunity.

In some embodiments, the condition, disease, or disorder is an inflammatory condition, disease, or disorder. In particular embodiments, the inflammatory condition, disease, or disorder is selected from the group consisting of an inflammatory airway disease, including an allergic airway inflammation, an ischemia-reperfusion injury, including cerebral ischemia and hepatic ischemia-reperfusion injury, sepsis, atherosclerosis, myocardial infarction, and inflammatory bowel disease.

In some embodiments, the condition, disease, or disorder is an infectious disease, including bacterial and viral infections. In particular embodiments, the infectious disease is a viral infection selected from the group consisting of HIV, Zika virus, rabies virus, Dengue virus, hepatitis C (HCV), hepatitis E (HEV), cytomegalovirus (HCMV), Newcastle disease virus (NDV), and Langat virus.

In other embodiments, the infectious disease is related to a toxin produced from a bacterial infection, including Shiga toxin released by Escherichia coli, and epsilon toxin, released by Clostridum perfringens.

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like

The term “combination” is used in its broadest sense and means that a subject is administered at least two agents, more particularly a compound of formula (I) and at least one other therapeutic agent. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the compounds of formula (I) described herein can be administered alone or in combination with adjuvants that enhance stability of the compounds of formula (I), alone or in combination with one or more therapeutic agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of a compound of formula (I) and at least one additional therapeutic agent can be varied so long as the beneficial effects of the combination of these agents are achieved. Accordingly, the phrase “in combination with” refers to the administration of a compound of formula (I) and at least one additional therapeutic agent either simultaneously, sequentially, or a combination thereof. Therefore, a subject administered a combination of a compound of formula (I) and at least one additional therapeutic agent can receive compound of formula (I) and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the compound of formula (I) and at least one additional therapeutic agent are administered simultaneously, they can be administered to the subject as separate pharmaceutical compositions, each comprising either a compound of formula (I) or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a compound of formula (I) and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

$${Q}_{a} / {Q}_{A} + {Q}_{b} / {Q}_{B} = Synergy Index ( SI )$$

wherein:

Q_{A} is the concentration of a component A, acting alone, which produced an end point in relation to component A;

Qₐ is the concentration of component A, in a mixture, which produced an end point;

Q_{B} is the concentration of a component B, acting alone, which produced an end point in relation to component B; and

Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

C. Methods for Inhibiting Neutral Sphingomyelinase 2 (nSMase2)

In some embodiments, the presently disclosed subject matter provides a method for inhibiting neutral sphingomyelinase 2 (nSMase2), the method comprising administering to a subject, cell, or tissue an amount of a compound of formula (I) effective to inhibit nSMase2.

As used herein, the term “inhibit,” and grammatical derivations thereof, refers to the ability of a presently disclosed compound, e.g., a presently disclosed compound of formula (I), to block, partially block, interfere, decrease, or reduce the activity of nSMase2. Thus, one of ordinary skill in the art would appreciate that the term “inhibit” encompasses a complete and/or partial decrease in the activity of nSMase2, e.g., a decrease by at least 10%, in some embodiments, a decrease by at least 20%, 30%, 50%, 75%, 95%, 98%, and up to and including 100%.

### D. Pharmaceutical Compositions and Administration

In another aspect, the present disclosure provides a pharmaceutical composition including one compound of formula (I) alone or in combination with one or more additional therapeutic agents in admixture with a pharmaceutically acceptable excipient. One of skill in the art will recognize that the pharmaceutical compositions include the pharmaceutically acceptable salts of the compounds described above. Pharmaceutically acceptable salts are generally well known to those of ordinary skill in the art, and include salts of active compounds which are prepared with relatively nontoxic acids or bases, depending on the particular substituent moieties found on the compounds described herein. When compounds of the present disclosure contain relatively acidic functionalities, base addition salts can be obtained by contacting the neutral form of such compounds with a sufficient amount of the desired base, either neat or in a suitable inert solvent or by ion exchange, whereby one basic counterion (base) in an ionic complex is substituted for another. Examples of pharmaceutically acceptable base addition salts include sodium, potassium, calcium, ammonium, organic amino, or magnesium salt, or a similar salt.

When compounds of the present disclosure contain relatively basic functionalities, acid addition salts can be obtained by contacting the neutral form of such compounds with a sufficient amount of the desired acid, either neat or in a suitable inert solvent or by ion exchange, whereby one acidic counterion (acid) in an ionic complex is substituted for another. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-toluenesulfonic, citric, tartaric, methanesulfonic, and the like. Also included are salts of amino acids such as arginate and the like, and salts of organic acids like glucuronic or galactunoric acids and the like (see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Accordingly, pharmaceutically acceptable salts suitable for use with the presently disclosed subject matter include, by way of example but not limitation, acetate, benzenesulfonate, benzoate, bicarbonate, bitartrate, bromide, calcium edetate, camsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000). In therapeutic and/or diagnostic applications, the compounds of the disclosure can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

Depending on the specific conditions being treated, such agents may be formulated into liquid or solid dosage forms and administered systemically or locally. The agents may be delivered, for example, in a timed- or sustained-slow release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances, such as saline; preservatives, such as benzyl alcohol; absorption promoters; and fluorocarbons.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.01 to 1000 mg, from 0.5 to 100 mg. from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, the bioavailability of the compound(s), the adsorption, distribution, metabolism, and excretion (ADME) toxicity of the compound(s), and the preference and experience of the attending physician.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

### E Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

While the following terms in relation to compounds of formula (I) are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

The terms substituted, whether preceded by the term “optionally” or not, and substituent, as used herein, refer to the ability, as appreciated by one skilled in this art, to change one functional group for another functional group on a molecule, provided that the valency of all atoms is maintained. When more than one position in any given structure may be substituted with more than one substituent selected from a specified group, the substituent may be either the same or different at every position. The substituents also may be further substituted (e.g., an aryl group substituent may have another substituent off it, such as another aryl group, which is further substituted at one or more positions).

Where substituent groups or linking groups are specified by their conventional chemical formulae, written from left to right, they equally encompass the chemically identical substituents that would result from writing the structure from right to left, e.g., —CH₂O— is equivalent to —OCH₂—; —C(═O)O— is equivalent to —OC(═O)—; —OC(═O)NR— is equivalent to —NRC(═O)O—, and the like.

When the term “independently selected” is used, the substituents being referred to (e.g., R groups, such as groups R₁, R₂, and the like, or variables, such as “m” and “n”), can be identical or different. For example, both R₁ and R₂ can be substituted alkyls, or R₁ can be hydrogen and R₂ can be a substituted alkyl, and the like.

The terms “a,” “an,” or “a(n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Unless otherwise explicitly defined, a “substituent group,” as used herein, includes a functional group selected from one or more of the following moieties, which are defined herein:

The term hydrocarbon, as used herein, refers to any chemical group comprising hydrogen and carbon. The hydrocarbon may be substituted or unsubstituted. As would be known to one skilled in this art, all valencies must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, isopropyl, cyclopropyl, allyl, vinyl, n-butyl, tert-butyl, ethynyl, cyclohexyl, and the like.

The term “alkyl,” by itself or as part of another substituent, means, unless otherwise stated, a straight (i.e., unbranched) or branched chain, acyclic or cyclic hydrocarbon group, or combination thereof, which may be fully saturated, mono- or polyunsaturated and can include di- and multivalent groups, having the number of carbon atoms designated (i.e., C₁₋₁₀ means one to ten carbons, including 1, 2, 3, 4, 5, 6, 7, 8, 9, and 10 carbons). In particular embodiments, the term “alkyl” refers to C₁₋₂₀ inclusive, including 1, 2, 3, 4, 5, 6, 7, 8, 9, 10, 11, 12, 13, 14, 15, 16, 17, 18, 19, and 20 carbons, linear (i.e., “straight-chain”), branched, or cyclic, saturated or at least partially and in some cases fully unsaturated (i.e., alkenyl and alkynyl) hydrocarbon radicals derived from a hydrocarbon moiety containing between one and twenty carbon atoms by removal of a single hydrogen atom.

Representative saturated hydrocarbon groups include, but are not limited to, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologs and isomers thereof.

“Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, cyano, and mercapto.

The term “heteroalkyl,” by itself or in combination with another term, means, unless otherwise stated, a stable straight or branched chain having from 1 to 20 carbon atoms or heteroatoms or a cyclic hydrocarbon group having from 3 to 10 carbon atoms or heteroatoms, or combinations thereof, consisting of at least one carbon atom and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two or three heteroatoms may be consecutive, such as, for example, —CH₂—NH—OCH₃ and —CH₂—O—Si(CH₃)₃.

As described above, heteroalkyl groups, as used herein, include those groups that are attached to the remainder of the molecule through a heteroatom, such as —C(O)NR′, —NR′R″, —OR′, —SR, —S(O)R, and/or —S(O₂)R′. Where “heteroalkyl” is recited, followed by recitations of specific heteroalkyl groups, such as —NR′R or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, unsubstituted alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl, and fused ring systems, such as dihydro- and tetrahydronaphthalene, and the like.

The term “cycloalkylalkyl,” as used herein, refers to a cycloalkyl group as defined hereinabove, which is attached to the parent molecular moiety through an alkylene moiety, also as defined above, e.g., a C₁₋₂₀ alkylene moiety. Examples of cycloalkylalkyl groups include cyclopropylmethyl and cyclopentylethyl.

The terms “cycloheteroalkyl” or “heterocycloalkyl” refer to a non-aromatic ring system, unsaturated or partially unsaturated ring system, such as a 3- to 10-member substituted or unsubstituted cycloalkyl ring system, including one or more heteroatoms, which can be the same or different, and are selected from the group consisting of nitrogen (N), oxygen (O), sulfur (S), phosphorus (P), and silicon (Si), and optionally can include one or more double bonds.

The cycloheteroalkyl ring can be optionally fused to or otherwise attached to other cycloheteroalkyl rings and/or non-aromatic hydrocarbon rings. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocyclic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidinyl, piperazinyl, indolinyl, quinuclidinyl, morpholinyl, thiomorpholinyl, thiadiazinanyl, tetrahydrofuranyl, and the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

As used herein the terms “bicycloalkyl” and “bicycloheteroalkyl” refer to two cycloalkyl or cycloheteroalkyl groups that are bound to one another. Non-limiting examples include bicyclohexane and bipiperidine.

An unsaturated hydrocarbon has one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl.”

More particularly, the term “alkenyl” as used herein refers to a monovalent group derived from a C₂₋₂₀ inclusive straight or branched hydrocarbon moiety having at least one carbon-carbon double bond by the removal of a single hydrogen molecule. Alkenyl groups include, for example, ethenyl (i.e., vinyl), propenyl, butenyl, 1-methyl-2-buten-1-yl, pentenyl, hexenyl, octenyl, allenyl, and butadienyl.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a monovalent group derived from a straight or branched C₂₋₂₀ hydrocarbon of a designed number of carbon atoms containing at least one carbon-carbon triple bond. Examples of “alkynyl” include ethynyl, 2-propynyl (propargyl), 1-propynyl, pentynyl, hexynyl, and heptynyl groups, and the like.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —CH₂CH₂CH₂CH₂—, —CH₂CH═CHCH₂—, —CH₂C_{S}CCH₂—, —CH₂CH₂CH(CH₂CH₂CH₃)CH₂—, —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons. Typically, an alkyl (or alkylene) group will have from 1 to 24 carbon atoms, with those groups having 10 or fewer carbon atoms being some embodiments of the present disclosure. A “lower alkyl” or “lower alkylene” is a shorter chain alkyl or alkylene group, generally having eight or fewer carbon atoms.

The term “heteroalkylene” by itself or as part of another substituent means a divalent group derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms also can occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—.

The term “aryl” means, unless otherwise stated, an aromatic hydrocarbon substituent that can be a single ring or multiple rings (such as from 1 to 3 rings), which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4- pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent forms of aryl and heteroaryl, respectively.

For brevity, the term “aryl” when used in combination with other terms (e.g., aryloxy, arylthioxy, arylalkyl) includes both aryl and heteroaryl rings as defined above. Thus, the terms “arylalkyl” and “heteroarylalkyl” are meant to include those groups in which an aryl or heteroaryl group is attached to an alkyl group (e.g., benzyl, phenethyl, pyridylmethyl, furylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). However, the term “haloaryl,” as used herein is meant to cover only aryls substituted with one or more halogens.

Where a heteroalkyl, heterocycloalkyl, or heteroaryl includes a specific number of members (e.g. “3 to 7 membered”), the term “member” refers to a carbon or heteroatom.

Further, a structure represented generally by the formula:

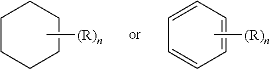

as used herein refers to a ring structure, for example, but not limited to a 3-carbon, a 4-carbon, a 5-carbon, a 6-carbon, a 7-carbon, and the like, aliphatic and/or aromatic cyclic compound, including a saturated ring structure, a partially saturated ring structure, and an unsaturated ring structure, comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 0 to 2 would comprise compound groups including, but not limited to:

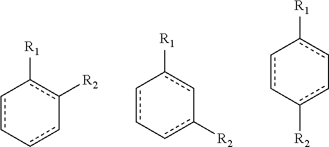

and the like.

A dashed line representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure.

The symbol ( [symbol] ) denotes the point of attachment of a moiety to the remainder of the molecule.

When a named atom of an aromatic ring or a heterocyclic aromatic ring is defined as being “absent,” the named atom is replaced by a direct bond.

Each of above terms (e.g. , “alkyl,” “heteroalkyl,” “cycloalkyl, and “heterocycloalkyl”, “aryl,” “heteroaryl,” “phosphonate,” and “sulfonate” as well as their divalent derivatives) are meant to include both substituted and unsubstituted forms of the indicated group. Optional substituents for each type of group are provided below.

Substituents for alkyl, heteroalkyl, cycloalkyl, heterocycloalkyl monovalent and divalent derivative groups (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R″′, —OC(O)R′, —C(O)R′, —CO₂R′, —C(O)NR′R″, —OC(O)NR′R″, —NR″C(O)R′, —NR′—C(O)NR″R″′, —NR″C(O)OR′, —NR—C(NR′R″)═NR″′, —S(O)R′, —S(O)₂R′, —S(O)₂NR′R″, —NRSO₂R′, —CN, CF₃, fluorinated C₁₋₄ alkyl, and —NO₂ in a number ranging from zero to (2m′+1), where m′ is the total number of carbon atoms in such groups. R′, R″, R″′ and R″″ each may independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. As used herein, an “alkoxy” group is an alkyl attached to the remainder of the molecule through a divalent oxygen. When a compound of the disclosure includes more than one R group, for example, each of the R groups is independently selected as are each R′, R″, R″′ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl groups above, exemplary substituents for aryl and heteroaryl groups (as well as their divalent derivatives) are varied and are selected from, for example: halogen, —OR′, —NR′R″, —SR′, —SiR′R″R″′, —OC(O)R′, —C(O)R′, —CO₂R′, —C(O)NR′R″, —OC(O)NR′R″, —NR″C(O)R′, —NR′—C(O)NR″R″′, —NR″C(O)OR′, —NR—C(NR′R″R″′)═NR″″, —NR—C(NR′R″)═NR″′—S(O)R′, —S(O)₂R′, —S(O)₂NR′R″, —NRSO₂R′, —CN and —NO₂, —R′, —N₃, —CH(Ph)₂, fluoro(C₁₋₄)alkoxo, and fluoro(C₁₋₄)alkyl, in a number ranging from zero to the total number of open valences on aromatic ring system; and where R′, R″, R″′ and R″″ may be independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the disclosure includes more than one R group, for example, each of the R groups is independently selected as are each R′, R″, R″′ and R″″ groups when more than one of these groups is present.

Two of the substituents on adjacent atoms of aryl or heteroaryl ring may optionally form a ring of the formula -T-C(O)—(CRR′)_{q}-U-, wherein T and U are independently —NR—, —O—, —CRR′— or a single bond, and q is an integer of from 0) to 3. Alternatively, two of the substituents on adjacent atoms of aryl or heteroaryl ring may optionally be replaced with a substituent of the formula -A-(CH₂)ᵣ-B, wherein A and B are independently —CRR′—, —O—, —NR—, —S—, —S(O)—, —S(O)₂—, —S(O)₂NR′— or a single bond, and r is an integer of from 1 to 4.

One of the single bonds of the new ring so formed may optionally be replaced with a double bond. Alternatively, two of the substituents on adjacent atoms of aryl or heteroaryl ring may optionally be replaced with a substituent of the formula —(CRR′)ₛ—X′—(C″R″′)_{d}—, where s and d are independently integers of from 0 to 3, and X′ is —O—, —NR′—, —S—, —S(O)—, —S(O)₂—, or —S(O)₂NR′—. The substituents R, R′, R″ and R″′ may be independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocylic, heterocyclic, or aromatic heterocyclic group as defined herein). As such, the term “acyl” specifically includes arylacyl groups, such as a 2-(furan-2-yl)acetyl)- and a 2-phenylacetyl group. Specific examples of acyl groups include acetyl and benzoyl. Acyl groups also are intended to include amides, —RC(═O)NR′, esters, —RC(═O)OR′, ketones, —RC(═O)R′, and aldehydes, —RC(═O)H.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, tert-butoxyl, and n-pentoxyl, neopentoxyl, n-hexoxyl, and the like.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl, i.e., C₆H₅—CH₂—O—. An aralkyloxyl group can optionally be substituted.

“Alkoxycarbonyl” refers to an alkyl-O—C(═O)— group. Exemplary alkoxycarbonyl groups include methoxy carbonyl, ethoxycarbonyl, butyloxycarbonyl, and tert-butyloxycarbonyl.

“Aryloxycarbonyl” refers to an aryl-O—C(═O)— group. Exemplary aryloxycarbonyl groups include phenoxy- and naphthoxy-carbonyl.

“Aralkoxycarbonyl” refers to an aralkyl-O—C(═O)— group. An exemplary aralkoxycarbonyl group is benzyloxycarbonyl.

“Carbamoyl” refers to an amide group of the formula —C(═O)NH₂. “Alkylcarbamoyl” refers to a R′RN—C(═O)— group wherein one of R and R′ is hydrogen and the other of R and R′ is alkyl and/or substituted alkyl as previously described. “Dialkylcarbamoyl” refers to a R′RN—C(═O)— group wherein each of R and R′ is independently alkyl and/or substituted alkyl as previously described.

The term carbonyldioxyl, as used herein, refers to a carbonate group of the formula —O—C(═O)—OR.

“Acyloxyl” refers to an acyl-O— group wherein acyl is as previously described.

The term “amino” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR′ wherein R′ is an alkyl group, as previously defined; whereas the term dialkylamino refers to a group having the structure —NR′R″, wherein R′ and R″ are each independently selected from the group consisting of alkyl groups. The term trialkylamino refers to a group having the structure —NR′R″R″′, wherein R′, R″, and R″′ are each independently selected from the group consisting of alkyl groups. Additionally, R′, R″, and/or R″′ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, isopropylamino, piperidino, trimethylamino, and propylamino.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH— group wherein acyl is as previously described. “Aroylamino” refers to an aroyl-NH— group wherein aroyl is as previously described.

The term “carbonyl” refers to the —C(═O)— group, and can include an aldehyde group represented by the general formula R—C(═O)H.

The term “carboxyl” refers to the —COOH group. Such groups also are referred to herein as a “carboxylic acid” moiety.

The term “cyano” refers to the —C≡N group.

The terms “halo,” “halide,” or “halogen” as used herein refer to fluoro, chloro, bromo, and iodo groups. Additionally, terms such as “haloalkyl,” are meant to include monohaloalkyl and polyhaloalkyl. For example, the term “halo(C₁₋₄)alkyl” is mean to include, but not be limited to, trifluoromethyl, 2,2,2-trifluoroethyl, 4-chlorobutyl, 3-bromopropyl, and the like.

The term “hydroxyl” refers to the —OH group.

The term “hydroxyalkyl” refers to an alkyl group substituted with an —OH group.

The term “mercapto” refers to the —SH group.

The term “oxo” as used herein means an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to a group of the formula —SH.

More particularly, the term “sulfide” refers to compound having a group of the formula —SR.

The term “sulfone” refers to compound having a sulfonyl group —S(O₂)R.

The term “sulfoxide” refers to a compound having a sulfinyl group —S(O)R

The term ureido refers to a urea group of the formula —NH—CO—NH₂.

Throughout the specification and claims, a given chemical formula or name shall encompass all tautomers, congeners, and optical- and stereoisomers, as well as racemic mixtures where such isomers and mixtures exist.

Certain compounds of the present disclosure may possess asymmetric carbon atoms (optical or chiral centers) or double bonds: the enantiomers, racemates, diastereomers, tautomers, geometric isomers, stereoisometric forms that may be defined, in terms of absolute stereochemistry, as (R)— or (S)— or, as D- or L- for amino acids, and individual isomers are encompassed within the scope of the present disclosure. The compounds of the present disclosure do not include those which are known in art to be too unstable to synthesize and/or isolate. The present disclosure is meant to include compounds in racemic, scalemic, and optically pure forms. Optically active (R)— and (S)—, or D- and L-isomers may be prepared using chiral synthons or chiral reagents, or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure: i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

Unless otherwise stated, structures depicted herein are also meant to include compounds which differ only in the presence of one or more isotopically enriched atoms. For example, compounds having the present structures with the replacement of a hydrogen by a deuterium or tritium, or the replacement of a carbon by ¹³C— or ¹⁴C— enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are encompassed within the scope of the present disclosure.

The compounds of the present disclosure may exist as salts. The present disclosure includes such salts. Examples of applicable salt forms include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent or by ion exchange. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

The term “protecting group” refers to chemical moieties that block some or all reactive moieties of a compound and prevent such moieties from participating in chemical reactions until the protective group is removed, for example, those moieties listed and described in T. W. Greene, P. G. M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups. For example, protective groups can be removed by acid, base, and hydrogenolysis. Groups such as trityl, dimethoxytrityl, acetal and tert-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as tert-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be blocked with oxidatively-removable protective groups such as 2,4-dimethoxy benzyl, while co-existing amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium(O)-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typical blocking/protecting groups include, but are not limited to the following moieties:

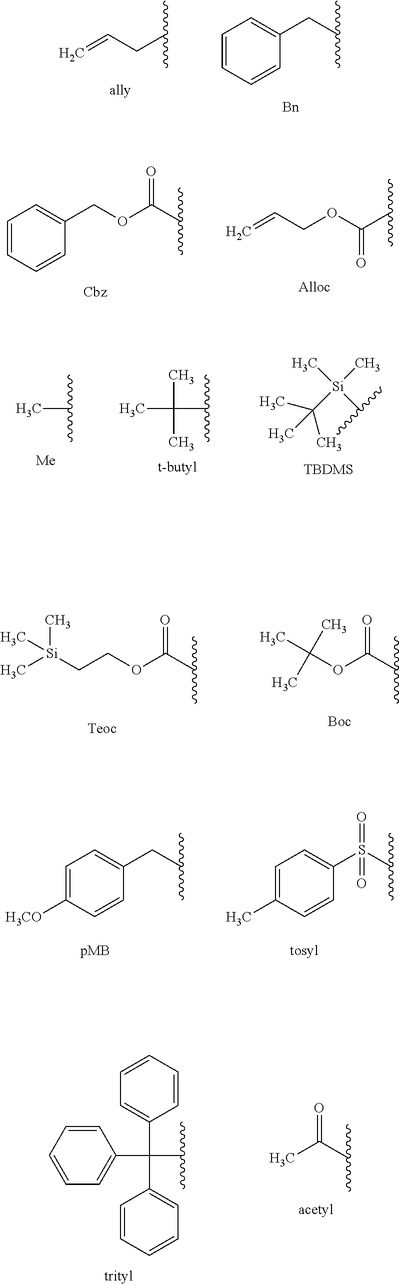

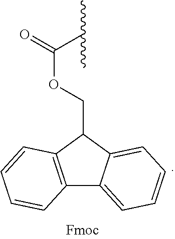 [SMILES: *C(=O)OCC1c2ccccc2-c2ccccc21]

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### nSMASE2 Inhibitor Prodrugs with Enhanced Oral and Brain Exposures

### 1.1 Overview

The presently disclosed subject matter provides the development and synthesis of neutral sphingomyelinases 2 (nSMase2) inhibitor prodrugs with enhanced oral availability and brain delivery. A human nSMase2 high throughput screen (HTS) of >350,000 compounds was carried out. Filtering and analysis of HTS hits led to the discovery 2,6-dimethoxy-4-(5-phenyl-4-(thiophen-2-yl)-1H-imidazol-2-yl) phenol (DPTIP), that was the most potent inhibitor of nSMase2 identified to date (IC₅₀=30 nM). Rojas et al., 2018. DPTIP was found to be selective and capable of dose-dependently inhibiting EV release in vitro. DPTIP, however, exhibited poor PK in mice with low exposures in plasma and brain (AUC_{brain}/AUCₚₗₐₛₘₐ<0.2) and rapid clearance (t_{1/2}<0.5 h). In an effort to address DPTIP's poor pharmacokinetics and enhance its delivery to the brain, various prodrugs were synthesized by chemical modification of the phenolic hydroxyl group. The metabolic stability and in vivo pharmacokinetics of these DPTIP prodrugs were evaluated. The prodrugs (e.g., P1, P2 and P5) demonstrated improved oral absorption and brain penetration. In brief, the presently disclosed subject matter demonstrates the prodrug approach to improve oral bioavailability and brain penetration of DPTIP.

### 1.2. Biological Methods

1.2.1 In vitro metabolic stability assessment. In vitro metabolic stability assessment was performed using mouse plasma, and liver homogenates. For the liver homogenates, washed tissues were diluted 10-fold in 0.1 M potassium phosphate buffer and homogenized using a probe sonicator. To evaluate the stability of the intact prodrug over time, each of the tissue matrix were aliquoted to 1 mL and then spiked with 10 μM of corresponding prodrugs followed by incubation in an orbital shaker at 37° C. for 1 h (in triplicate). Sample from each incubation at predetermined time points (0 min, and 1 h) was quenched with three volumes of acetonitrile containing the internal standard (IS: losartan: 0.5 μm). Samples were vortex-mixed for 30 secs and centrifuged at 10,000 RPM for 10 min at 4° C. Disappearance of the intact compound was measured using liquid chromatography tandem mass spectrometry (LC-MS/MS).
1.2.2 In vivo pharmacokinetic studies in mice. Male CD-1 mice (n=3) were used to study the pharmacokinetic profiles of representive prodrug compounds. All compounds were administered orally at a dose of 10 mg/kg equivalent of DPTIP using a formulation of 5% DMSO, 10% Tween in PBS. Blood and brain were collected after sacrificing the mice at predetermined time points. Plasma was harvested from blood by centrifugation at 3,000 RPM for 15 min and stored at −80° C. Brain tissues were harvested following blood collection and immediately snap frozen in liquid nitrogen and stored at −80° C. until LC-MS/MS analysis. Calibration standards were prepared using naïve mouse plasma or brain with additions of respective DPTIP Prodrugs and DPTIP. For quantifying the intact prodrugs and released DPTIP in the pharmacokinetic samples, plasma samples (20 μL) were processed using a single liquid extraction method by addition of 100 μL of acetonitrile containing internal standard (losartan: 0.5 μM), followed by vortex-mixing for 30 s and then centrifugation at 10,000 RPM for 10 min at 4° C. Brain tissues were diluted 1:5 w/v with acetonitrile containing losartan (0.5 μm) and homogenized, followed by vortex-mixing and centrifugation at 10,000 RPM for 10 min at 4° C. A 50 μL aliquot of the supernatant was diluted with 50 μL of water and transferred to 250 μL polypropylene autosampler vials sealed with teflon caps. Then, 2 μL of the sample was injected into the LC-MS/MS system for analysis.

### 1.3 Results

1.3.1 Prodrugs showed improved stability in mouse plasma and liver homogenates. We evaluated the metabolic stability of representative prodrug compounds using mouse plasma and liver homogenates. Compounds P1, P15, P16, P6, 5′, P14 and P5 were found to be stable in mouse plasma with >75% intact prodrug remaining after 1 hour of incubation at 37° C. Compounds P2, P3 and P4 were found unstable (<30% remaining at 1 h) and P17 was moderately stable (approximately 50% remaining at 1h) in mouse plasma. In mouse liver homogenates, compounds P1, P6 and P5 were stable with >75% intact prodrug remaining after 1 hour of incubation at 37° C. Compounds 5′ and P14 were moderately stable (>50% remaining at 1 h) and p15, P16, P2, P3, P4 and P17 were completely unstable (<25% remaining at 1 h).
1.3.2 Prodrugs (P1, P2 and P5) improved oral absorption and brain penetration compared to DPTIP. The in vivo pharmacokinetics of representative prodrug compounds in mice were evaluated. Mice were dosed (10 mg/kg equivalent of DPTIP) and plasma and brain levels of intact prodrugs and released DPTIP were measured at predetermined time points. Of all the compounds tested, compound 6 showed higher levels of DPTIP release in plasma (Cₘₐₓ˜2.8 μM) and brain (Cₘₐₓ˜130 nM) although the brain levels were higher than DPTIP (50 nM vs approximately 150 nM), brain-to-plasma ratio was unchanged (approximately 0.1). Compound P5 showed improved oral absorption (10×) as intact with a plasma Cₘₐₓ of 4.3 μM as compared to DPTIP (plasma Cₘₐₓ 400 nM). Compound P5 also showed sustained level of released DPTIP in the brain (approximately 45 nM up-to 2 h). However, the brain-to-plasma ratio was not enhanced versus DPTIP (approximately 0.1). In the case of released DPTIP from the prodrugs, Compound P1 showed improved brain penetration with a brain Cₘₐₓ of 72.34 nM as compared to 50.8 nM for DPTIP. The brain-to-plasma ratio of released DPTIP from compound P1 was 0.69 as compared to 0.1 of DPTIP.

Owing to the promising outcomes of compounds P1 and P2 in the initial pharmacokinetic analysis, these compounds were selected for a detailed pharmacokinetic analysis in mice. Compound P1 exhibited a 3.7-fold increase in brain levels (187.1 versus 52.8 pmol.h/g) and a 7-fold improvement in brain penetration index (AUC_{brain/plasma} ratio 0.70 vs 0.1) with a remarkable improvement in apparent half-life (t_{1/2}=3 h vs <0.5 h) when compared to equimolar DPTIP. Compound P2 also showed a remarkable 5- and 2-fold enhancement in DPTIP plasma concentration (AUC₀₋ₜ=1495 versus 270 pmol.h/mL) and brain exposure (AUC₀₋ₜ of 105 vs 52.8 pmol.h/g) versus equimolar DPTIP. The detailed pharmacokinetic parameters of Compound P1 and Compound P2 are provided in Table 1-1.

TABLE 1-1
Pharmacokinetic parameters of Compound P1 and P2

| Dose: 10 | | Cₘₐₓ | Tₘₐₓ | AUC | t_{1/2} | MRT | Brain:Plasma |
|---|---|---|---|---|---|---|---|
| mg/kg eqv | Tissue | (pmol/mL) | (min) | (pmol · h/mL) | (h) | (h) | ratio |
| Compound P1 | Plasma | 546.1 ± 25.8 | 1 | 804.6 | 2.45 | 3.39 | 0.6 |
| (Intact) | Brain | 105.5 ± 11.6 | 1 | 477.1 | 4.37 | 2.12 | |
| Compound P1 | Plasma | 148.9 ± 9.9 | 1 | 273.4 | 2.38 | 2.67 | 0.7 |
| (DPTIP | Brain | 40.3 ± 4.5 | 1 | 187.1 | 5.3 | 3.02 | |
| release) | | | | | | | |
| Compound P2 | Plasma | 51.1 ± 4.6 | 15 | 7.04 | — | — | 0.23 |
| (intact) | Brain | 9.8 ± 0.6 | 15 | 1.66 | — | — | |
| Compound P2 | Plasma | 2636.44 ± 224.89 | 30 | 1495.16 | 0.81 | 0.88 | 0.07 |
| (DPTIP | Brain | 122.4 ± 16.8 | 30 | 105.46 | 1.09 | 1.4 | |
| Release) | | | | | | | |

### Example 2

### Discovery of Orally Bioavailable and Brain Penetrable Prodrugs of the Potent Nsmase2 Inhibitor DPTIP

### 2.1 Overview

Extracellular vesicles (EVs) can carry pathological cargo and play an active role in disease progression. Neutral Sphingomyelinase-2 (nSMase2) is a critical regulator of EV biogenesis, and its inhibition has shown protective effects in multiple disease states. 2,6-Dimethoxy-4-(5-phenyl-4-thiophen-2-yl-1H-imidazol-2-yl)-phenol (DPTIP) is one of the most potent (IC₅₀=30 nM) inhibitor of nSMase2 discovered to-date. DPTIP, however, exhibits poor oral pharmacokinetics (PK), limiting its clinical development. To overcome DPTIP's PK limitations, a series of prodrugs was synthesized by masking its phenolic hydroxyl group. When administered orally, the best prodrug (P18) with a bipiperidinyl-promoiety exhibited greater than 4-fold higher plasma (AUC₀₋ₜ=1047 pmol.h/mL) and brain exposures (AUC₀₋ₜ=247 pmol.h/g) versus DPTIP; and a significant enhancement of DPTIP half-life (2 h vs. approximately 0.5 h). In a mouse model of acute brain injury, DPTIP released from P18 significantly inhibited IL-1β-induced EV release into plasma and attenuated nSMase2 activity. This example provides the discovery of a DPTIP-prodrug with potential for clinical translation.

### 2.2 Background

Extracellular vesicles (EVs) are small vesicular carriers that contain a variety of cargo including proteins, nucleic acids, and bioactive lipids. Tallon et al., 2021. EVs are shed from cells in response to various stimuli and they regulate a large variety of intercellular communication both in physiologic and pathologic conditions. Ibrahim and Marban, 2016; Raposo and Stoorvogel, 2013; Weidle et al., 2017. Cumulative evidence suggests that under disease conditions, EVs can carry pathological cargo and play an active role in disease propagation. Ibrahim and Marban, 2016; Raposo and Stoorvogel, 2013; Weidle et al., 2017. A member of the sphingomyelinase (SMase) enzyme family, neutral sphingomyelinase2 (nSMase2), catalyzes the hydrolysis of sphingomyelin, Luberto et al., 2002, to ceramide, which is a major regulator of the biogenesis and release of EVs. Trajkovic et al., 2008. Although temporary increases in nSMase2 activity are observed in normal physiological processes, chronic increase of nSMase2 activity has been implicated in multiple pathological disorders including brain inflammation, Dickens et al., 2017, cancer metastasis, Kosaka et al., 2013, amyloid deposition, Dinkins et al., 2016; Dinkins et al., 2014; Sardar Sinha et al., 2018, tau protein propagation, Asai et al., 2015, and HIV infection. Barclay et al., 2017; Dalvi et al, 2017; Shukla et al., 2012; Sun et al., 2017. Indeed, genetic and pharmacological inhibition of nSMase2 has been reported to reduce EV levels and improve disease pathology. Tallon et al., 2021. Given this, there is a rising interest in developing clinically viable inhibitors for nSMase2

Unfortunately, there are currently no clinically useful nSMase2 inhibitors. Current inhibitors are weak (μM-mM) with poor physicochemical properties and/or limited brain penetration. Our laboratory previously reported DPTIP (2,6-dimethoxy-4-(5-phenyl-4-thiophen-2-yl-1H-imidazol-2-yl)-phenol), as one of the most potent inhibitors of nSMase2 identified to date (IC₅=30 nM). Rojas et al., 2018; Stepanek et al., 2019. Its IC₅₀ is 10- to 160-fold lower than the known inhibitors. Luberto et al., 2002; Figuera-Losada et al., 2015; Rojas et al., 2019. DPTIP was found to be selective, and capable of dose-dependently inhibiting EV release in vitro and in vivo (when delivered systemically). DPTIP, however, exhibits poor pharmacokinetics and rapid clearance (t_{1/2}<0.5 h). Structural modifications on the DPTIP scaffold did not lead to substantial improvements. Stepanek et al., 2019.

### 2.3 Scope

Given the excellent potency, selectivity, and stability of DPTIP, we aimed to advance DPTIP by optimizing its PK profile using prodrug strategies. Aungst and Hussain, 1987; Hussain et al., 2002; Kao et al., 2000. Notably, prodrug approaches are common in drug development; about 10-12% of the approved drugs are prodrugs, and of these, most are designed to enhance the permeation of the parent drug, Rautio et al., 2018, through biological membranes. Our group has demonstrated a history of success in modulating PK properties of drug candidates by employing prodrug strategies; these include, improving solubility, Zimmermann et al., 2018, enhancing permeability across biological membranes, Dash et al., 2019, brain penetration, Rais et al., 2016, and tumor targeting. Tenora et al., 2019.

This example provides the synthesis, in vivo PK evaluation, and target engagement of DPTIP prodrugs that were designed based on previous successes in improving the PK properties of poorly permeable molecules. Amongst the series of DPTIP-prodrugs synthesized and characterized, we identified P18, a prodrug with a bipiperidinyl-based promoiety on the phenolic site of DPTIP. P18 exhibited an excellent PK profile compared to DPTIP. Moreover, orally administered P18 significantly inhibited IL-1β-induced EV release by inhibition of brain nSMase2 activity. Thus, P18 is a novel DPTIP prodrug that can aid in clinical translation of this class of inhibitors.

### 2.4 Results and Discussion

### 2.4.1 Chemistry

We have previously evaluated PK of DPTIP analogs in rodents and reported O-glucuronidation at the phenolic hydroxyl group as their primary path of metabolism. Stepanek et al., 2019. This glucuronidation makes DPTIP susceptible to rapid clearance leading to poor half-life. One of our first strategies, therefore, was to deter glucuronidation by derivatization of the phenolic hydroxyl site of DPTIP; thus, improving its half-life. We further strategized that the promoiety architecture could be modulated to improve oral absorption and/or brain penetration of DPTIP. Pursuant to this strategy, we initially synthesized DPTIP prodrugs supporting alkyl chain promoieties coupled either by ester or carbamate linkages (P1-P13, and P18). These also included the various piperidine promoieties as well as the piperidinopiperidine promoiety that has previously been employed to mask the phenolic site of SN-38, an FDA approved drug for the treatment of colorectal cancers (CPT-11; irinotecan hydrochloride). Slatter et al., 2000. With phenolic site masked, the in vivo metabolism of the prodrugs was primarily dominated by carboxylesterase enzymes which cleave both the ester or carbamate linkage of the inactive prodrug to release DPTIP. The rate of metabolic release of DPTIP is thus controlled by the stability of the linker which can be tuned via ester/carbamate linkage in addition to the steric hindrance imparted by the promoiety.

DPTIP is lipophilic (cLogP=4.72) which limits its solubility in formulations required for oral dosing. To overcome this physicochemical limitation, our second strategy included designing phosphate esters (P14-P16) to increase hydrophilicity as well as polar surface of DPTIP. We aimed to achieve a hydrophilic/lipophilic balance and enhance oral exposures by improving dissolution rate of DPTIP. This strategy has previously been reported to improve solubility of miproxifene via its phosphorylation. Heimbach et al., 2003. We also have previously reported a mebendazole prodrug where its phosphate ester displayed a 2.2-fold higher plasma and 1.7-fold higher brain exposures when tested in rodents. Similarly, in dogs, phosphate ester of mebendazole resulted in a 3.8-fold higher plasma exposures. Zimmermann et al, 2018. P15 and P16 were designed to further modulate the stability of linkages of phosphate prodrugs in addition to optimizing their physicochemical properties. Overall, these strategies were anticipated to improve solubility of DPTIP-prodrugs and enhance their absorption, following activation in intestinal brush border or liver where resident phosphatase enzymes would trigger DPTIP release.

Finally, we also synthesized a N-methyl-1,4-dihydronicotinic ester prodrug of DPTIP (P17) designed to passively absorb through GI tract and eventually through the blood brain barrier (BBB). The prodrug would then acquire a positive charge in the brain due to aromatization, forming a pyridinium salt impermeable to BBB. This ‘trapped’ prodrug accumulating in the brain then is expected to release DPTIP. We describe the synthesis and characterization of the 18 DPTIP prodrugs below.

### 2.4.2 Synthesis

DPTIP was synthesized using our previously reported methods. Rojas et al., 2018; Stepanek et al., 2019. Prodrugs P1-P13 were synthesized in a single step reaction using DPTIP and respective amines as the starting material in the presence of triphosgene and Hünig's base (DIPEA) to form either esters or carbamates (Scheme-1).

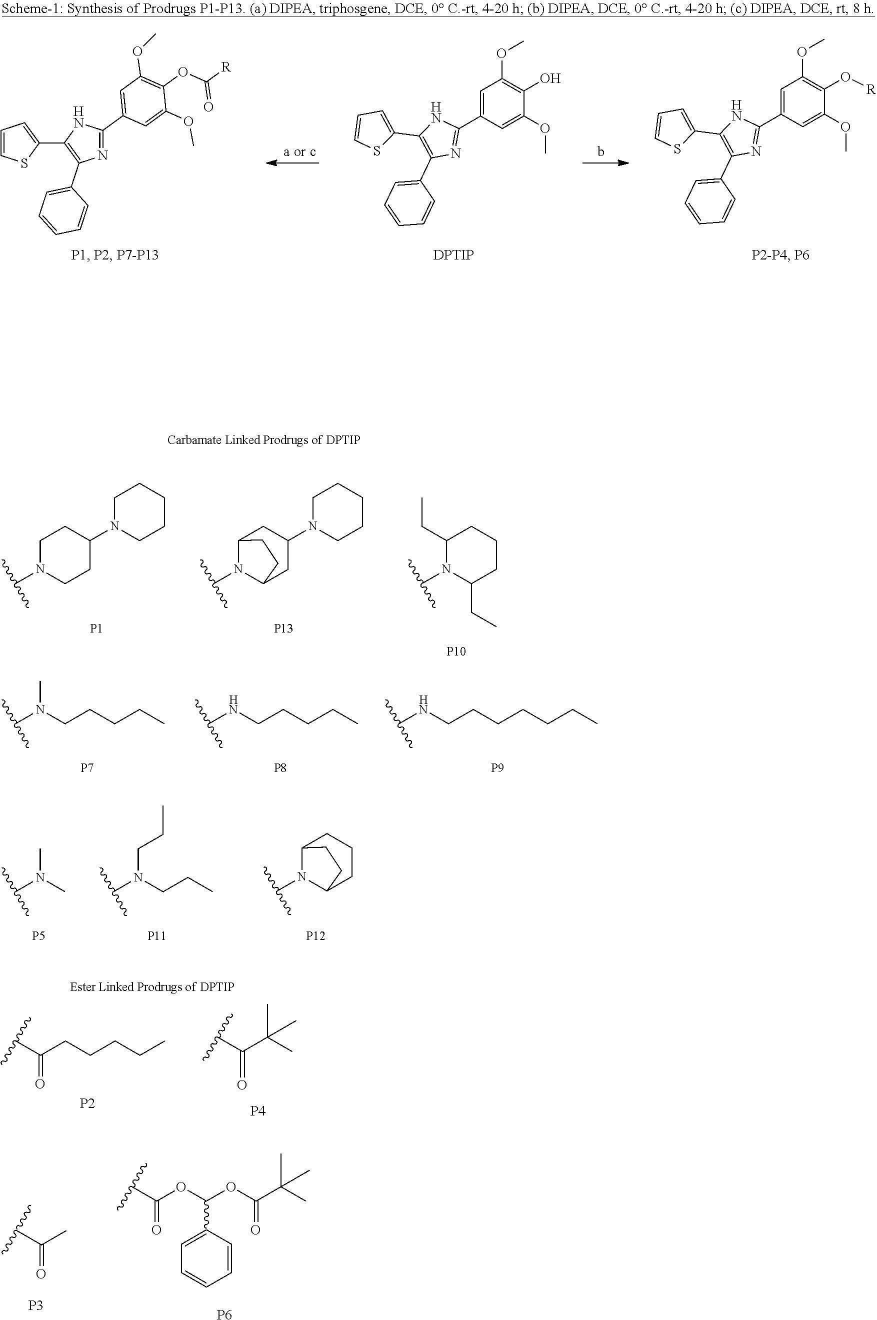

Briefly, DPTIP was treated with triphosgene in the presence of DIPEA in dichloroethane at 0° C. and stirred for 1 h. The amine was then added to this mixture and the reaction was periodically monitored via thin layer chromatography (TLC) for completion (4-20 h). If carbonyl chlorides of the promoieties were available, the reaction was carried out under similar conditions as described above but in the absence of triphosgene. Detailed reaction conditions for synthesis of all prodrugs are provided in Section 2.6 herein below.

The phosphoester of DPTIP P14 was synthesized by addition of phosphoryl chloride and DPTIP to a mixture of DIPEA in DCE followed by potassium carbonate treatment (Scheme-2). The reaction was stirred until completion and then crude mixture purified using preparative column chromatography. P15 and P16 were obtained via the intermediate 4, which was synthesized by reaction of DPTIP with t-butyl-protected chloromethyl phosphate in presence of cesium carbonate and sodium iodide (Scheme-2).

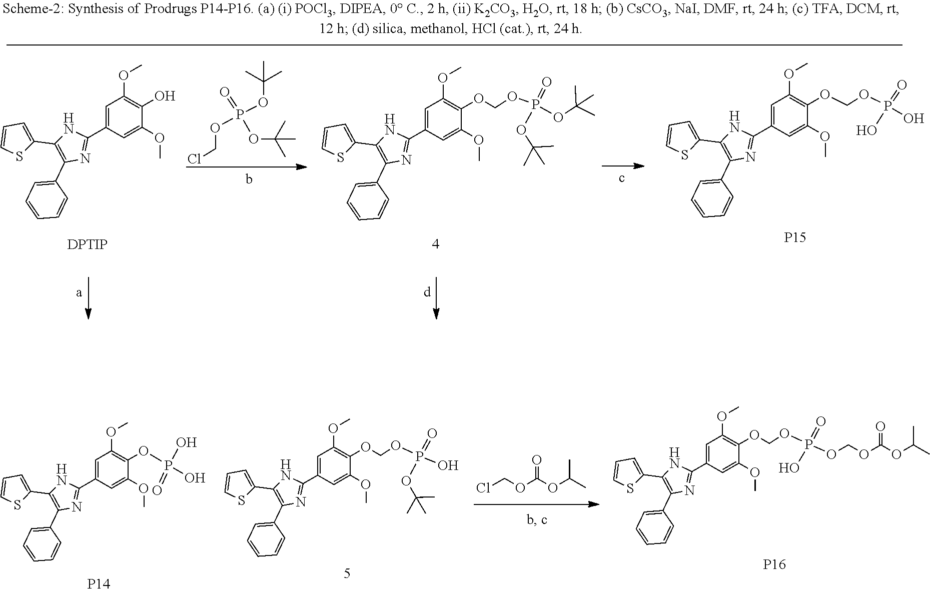

Intermediate 4 then was treated with trifluoroacetic acid (TFA) to obtain P15. To obtain P16, intermediate 4 was first treated with silica and catalytic HCl for 24 h at room temperature, when partially deprotected intermediate 5 was obtained. This was then treated with chloromethyl isopropyl carbonate in presence of cesium carbonate and sodium iodide, followed by deprotection of the t-butyl ester using trifluoroacetic acid to reveal P16. P17 was obtained in three steps via Steglich esterification of DPTIP with nicotinic acid followed by N-alkylation with methyl iodide (Scheme-3).

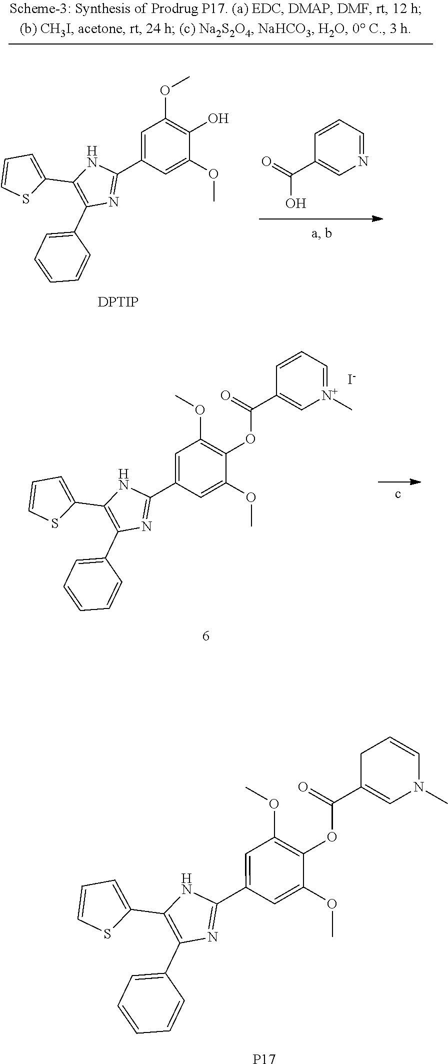

In the final step the resulting pyridinium salt is treated with sodium dithionite to reveal P17, containing N-methyl-1,4-dihydropyridine moiety. P18 was synthesized via carbamation of DPTIP and intermediate 8 using the conditions described earlier (P1-P13). 2′,6′-Diethyl-1,4′-bipiperidine (8) was prepared in a 3-step synthetic procedure starting with 3-oxopentanedioic acid and benzylamine in the presence of propionaldehyde. The cyclized intermediate 7 was then coupled with piperidine in the presence of sodium cyanoborohydride followed by removing of benzyl group with palladium catalyst and hydrogen gas to reveal intermediate 8 (Scheme-4).

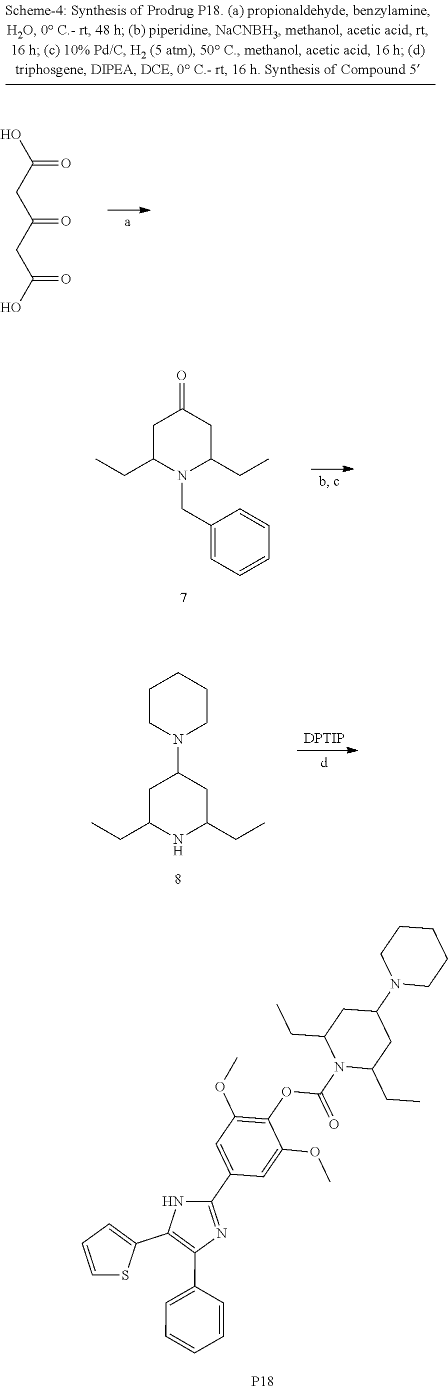

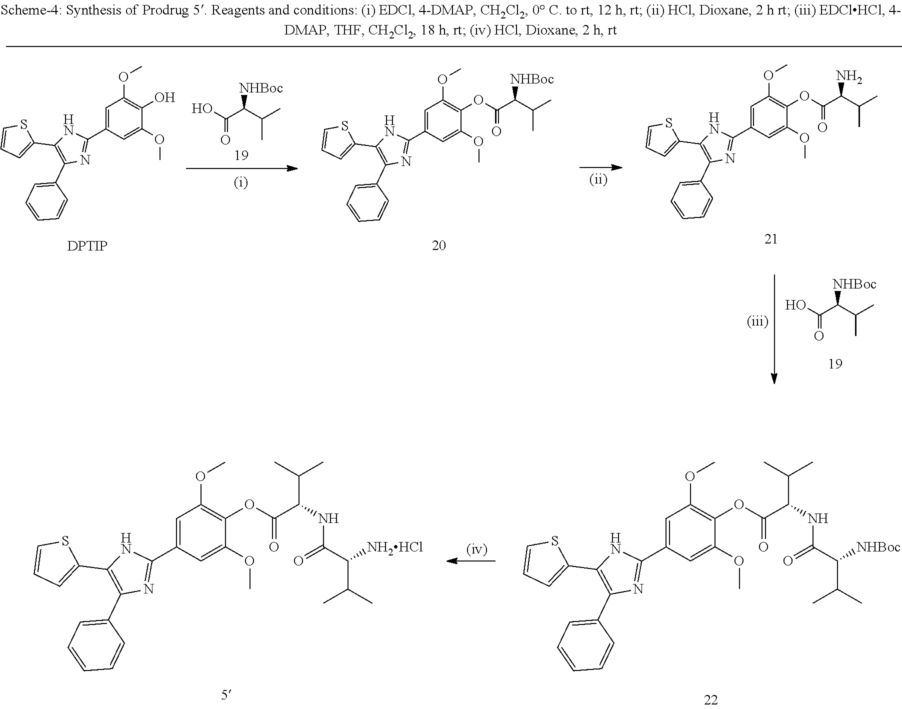

### 2.4.3 Prodrugs Exhibited Broad Range of Lipophilicity and Metabolic Stability

Eighteen prodrugs of DPTIP were designed, synthesized, and assessed with the goal to enhance metabolic stability as well as to the facilitate oral absorption and brain penetration. First, cLogP values were obtained in silico using ChemDraw Professional 20.1 software (Table 2-1). The prodrugs showed varying degrees of lipophilicity (cLogP from 2.99 to 7.36) compared to DPTIP (cLogP=4.72). As expected, a majority of the prodrugs containing alkyl, piperidine, and piperidinopiperidine promoieties exhibited an increase in the cLogP (from 4.84 to 7.36) mirroring the lipophilic character of the promoieties. In contrast, compounds containing phosphate ester promoieties (P14 and P15) exhibited a decrease in lipophilicity (cLogP ranged from 2.99 to 3.18). The decrease in cLogP for these compounds was attributed to the charged nature of the promoieties for enhanced dissolution. P16 containing a phospho-ester with an isopropyloxycarbonyloxymethyl (POC) group depicted a higher cLogP due to masking of one of the charges. Owing to the low solubility and permeability of DPTIP, the 18 prodrugs were designed with a broad range of chemical groups to provide improvement in either solubility or permeability with the ultimate goal of enhancing oral absorption.

TABLE 2-1
Lipophilicity and metabolic stabilities of DPTIP prodrugs P1-P18

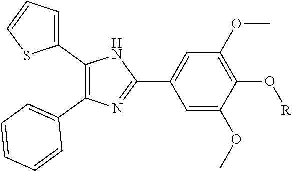

| | | | Stability in mouse | | |
|---|---|---|---|---|---|
| | | | (% remaining at 1h) | | |
| | | | Mean + Std Error | | |
| Compound | R | cLogP | Plasma | Liver | Brain |
| DPTIP | —H | 4.72 | 103 ± 2 | 98 ± 6 | 89 ± 4 |
| P1 | 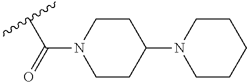 [SMILES: *C(=O)N1CCC(N2CCCCC2)CC1] | 5.2 | 90 ± 2 | 85 ± 3 | 48 ± 2 |
| P2 | 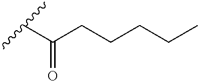 [SMILES: *C(=O)CCCCC] | 6.52 | 13 ± 1 | 1 ± 0 | 63 ± 1 |
| P3 | 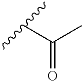 [SMILES: *C(C)=O] | 4.41 | 28 ± 2 | 0 ± 0 | 6 ± 0 |
| P4 | 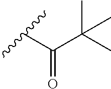 [SMILES: *C(=O)C(C)(C)C] | 5.65 | 1 ± 0 | 0 ± 0 | 93 ± 1 |
| P5 | 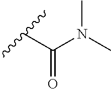 [SMILES: *C(=O)N(C)C] | 4.06 | 96 ± 3 | 100 ± 1 | 100 ± 2 |
| P6 | 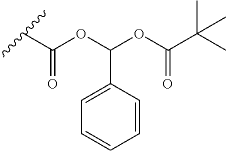 [SMILES: *C(=O)OC(OC(=O)C(C)(C)C)c1ccccc1] | 6.92 | 83 ± 3 | 80 ± 4 | 69 ± 2 |
| P7 | 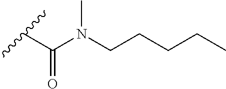 [SMILES: *C(=O)N(C)CCCCC] | 6.19 | 50 ± 2 | 85 ± 3 | 86 ± 1 |
| P8 | 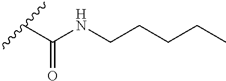 [SMILES: *C(=O)NCCCCC] | 5.97 | 60 ± 6 | 83 ± 2 | 68 ± 1 |
| P9 | 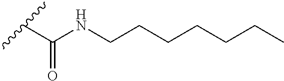 [SMILES: *C(=O)NCCCCCCC] | 7.02 | 60 ± 4 | 97 ± 4 | 84 ± 4 |
| P10 | 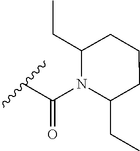 [SMILES: *C(=O)N1C(CC)CCCC1CC] | 7.36 | 78 ± 5 | 51 ± 1 | 90 ± 1 |
| P11 | 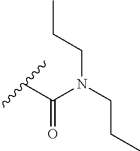 [SMILES: *C(=O)N(CCC)CCC] | 6.19 | 99 ± 2 | 62 ± 4 | 95 ± 4 |
| P12 | 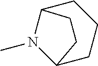 | 5.58 | 85 ± 9 | 95 ± 3 | 76 ± 3 |
| P13 | 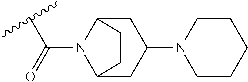 [SMILES: *C(=O)N1C2CCC1CC(N1CCCCC1)C2] | 5.54 | 87 ± 2 | 78 ± 4 | 84 ± 1 |
| P14 | 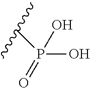 [SMILES: *P(=O)(O)O] | 3.18 | 91 ± 4 | 55 ± 1 | 102 ± 2 |
| P15 | 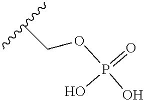 [SMILES: *COP(=O)(O)O] | 2.99 | 96 ± 6 | 21 ± 0 | 1 ± 0 |
| P16 | 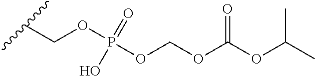 [SMILES: *COP(=O)(O)OCOC(=O)OC(C)C] | 5.97 | 100 ± 3 | 11 ± 0 | 79 ± 2 |
| P17 | 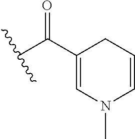 [SMILES: *C(=O)C1=CN(C)C=CC1] | 4.84 | 52 ± 2 | 22 ± 1 | 46 ± 0 |
| P18 | 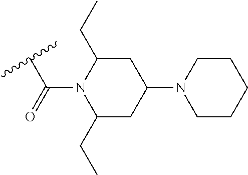 [SMILES: *C(=O)N1C(CC)CC(N2CCCCC2)CC1CC] | 5.55 | 92 ± 7 | 100 ± 3 | 100 ± 1 |
| 5′ | 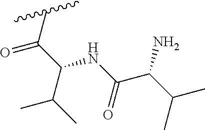 [SMILES: *C(=O)[C@H](NC(=O)[C@H](N)C(C)C)C(C)C] | 5.30 | 79 ± 4 | 64 ± 0 | 45 ± 2 |

All synthesized prodrugs were next screened for in vitro stability in carboxylesterase 1 knockout (CES1^{-/-}) mouse plasma, as well as CES1^{-/-} mouse brain and liver homogenates (Table 2-1). We have previously reported that CES1^{-/-} mouse model recapitulates human prodrug metabolism. Tenora et al., 2019. These studies were conducted to evaluate the activation of the prodrugs and their conversion to DPTIP in these highly metabolically-active sites. The results from these in vitro assays were crucial for the selection of prodrugs with low susceptibility to systemic metabolism thereby enabling biodistribution of the intact prodrugs to the target site, specifically the CNS. DPTIP can then be released from these prodrugs by the activity of local, endogenous enzymes. The most desirable prodrugs showed considerable metabolic stability in plasma, and liver homogenates but modest metabolism and conversion in the brain homogenates. P1, P5, P6, and P10-16, P18 were found to be stable in CES1^{-/-} mouse plasma with >75% intact prodrug remaining after 1 hour of incubation at 37° C. P7, P8, P9 and P17 were moderately stable (˜50-60% remaining at 1h) and P2, P3, and P4 were unstable (<30% remaining at 1 h). In mouse liver homogenates, P1, P5, P6, P12, P13, and P18 were metabolically stable with >75% intact prodrug remaining after 1 hour of incubation at 37° C. P10, P11, and P14 were moderately stable (>50% remaining at 1 h) and P2, P3, P4, P15, P16, and P17 were highly metabolized (<25% remaining at 1 h) in liver homogenates. In mouse brain homogenates, P4, P5, P14, P16, and P18 were found to be metabolically stable with >75% intact prodrug remaining after 1 hour of incubation at 37° C. P1, P2, P6, and P17 were moderately stable (˜50% or higher remaining at 1 h) and P3 and P15 were completely metabolized in brain homogenates.

In general, compounds containing ester promoieties showed highest metabolism in plasma and liver as these are likely cleaved by the ubiquitous carboxylesterase enzymes highly abundant at these sites. One exception to this was P6, which showed modest metabolic stability against the esterase cleavage, likely due to the steric hinderance of the phenyl and pivalate promoieties. In contrast, prodrugs comprising the carbamate linkage (either alkyl carbamate or piperidinopiperidine promoiety) demonstrated higher metabolic stability in plasma and liver homogenates. Interestingly, a subset of the carbamate prodrugs (P1, P7, P8, P12) showed considerable metabolism in the brain homogenates. Lastly, the compounds containing the phospho-esters (P15, P16) were stable in plasma. However, P15 was completely metabolized and P16 (phosphoester with POC group) was moderately metabolized in brain homogenates. Given the broad stability profile for prodrugs P1-P18 we subsequently evaluated them in a single time point PK in plasma and brain; and selected the best of those for time-dependent PK evaluation.

### 2.4.4 Prodrugs Improved Oral Absorption and Brain Penetration Compared to DPTIP

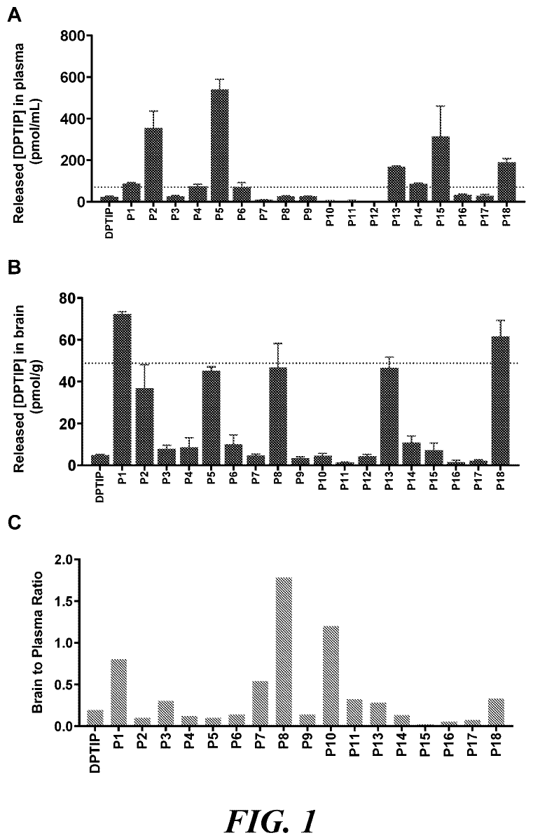

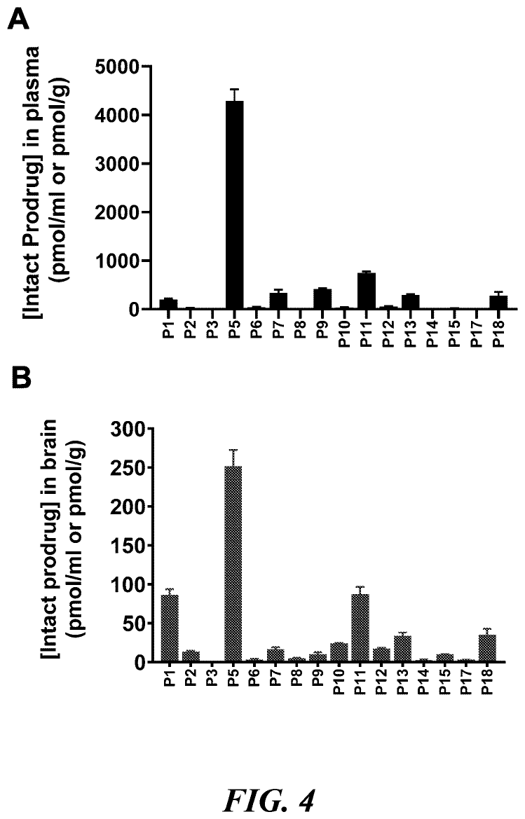

Mice dosed orally (10 mg/kg equivalent to DPTIP) were sacrificed 2 h after dose; plasma and brain were collected to measure the levels of intact prodrug and released DPTIP in these matrices. 2 h time point was selected because DPTIP was expected to show low levels due to its rapid clearance and only prodrugs which showed improvement were advanced for time-dependent evaluation. The results from the PK analysis of DPTIP prodrugs are shown in FIG. 1A-FIG. 1C and FIG. 4. Amongst all the prodrugs tested, P2 with the hexanoate ester showed an increase in DPTIP release (FIG. 1A and FIG. 4) in plasma (355 nM) and brain (36.8 nM); and although the brain levels were higher than DPTIP (36.8 nM vs. 4.9 nM), brain-to-plasma ratio remain unchanged (approximately 0.1). The higher level of DPTIP released in plasma was attributed to high lipophilicity imparted by the hexanoate ester as well as instability of the ester in plasma and liver due to the presence of an ester linkage. P5, a prodrug containing dimethyl carbamate promoiety, also showed a notable improvement in oral absorption/ viz. 4.3 μM intact P5 (FIGS. 4) and 540.9 nM released DPTIP was measured in plasma. In contrast, DPTIP when dosed orally at 10 mg/kg showed poor levels in plasma (approximately 23 nM). However, similar to P2, brain penetration index of P5 was comparable to DPTIP (approximately 0.1). P1, a piperidinopiperidine conjugate of DPTIP, released 72.34 nM DPTIP in brain at 2h post-dose, thus outperforming DPTIP at same dose level (4.9 nM) (FIG. 1A-FIG. 1B). Notably, P1 showed a remarkable improvement in the brain-to-plasma ratio of released DPTIP (approximately 0.7) as compared DPTIP (approximately 0.1). P18, that was designed combining the chemical attributes of P1 and P2, with a diethyl-piperidinopiperidine promoiety, exhibited desirable PK with higher plasma (189.3 nM vs. 23 nM), and brain (61.5 nM vs. 4.9 nM) levels as well as higher brain-to-plasma ratio of 0.33. Amongst the compounds evaluated, we selected those that showed high release of DPTIP in both plasma (>3-fold) and brain (>10 fold) following oral administration and/or improvement in its brain/plasma ratio (e.g. P1). The single time point PK helped to exclude prodrugs (P2-P7, P9-P12, and P14-P17) that were worse than DPTIP in improving the brain and plasma levels of DPTIP and/or its brain penetration index. Among the remaining compounds, P1 and P18 demonstrated the highest improvement in both of these parameters, and were advanced for further time-dependent PK evaluation.

### 2.4.5 P18 Exhibited Approximately 5-Fold Higher Exposures in Brain and Plasma and Improved Brain Penetration Index Following PO Administration in Mice as Compared to DPTIP

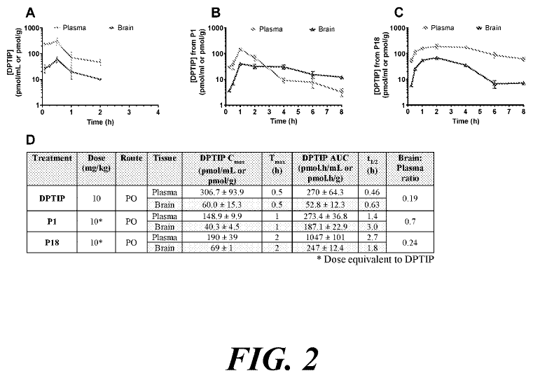

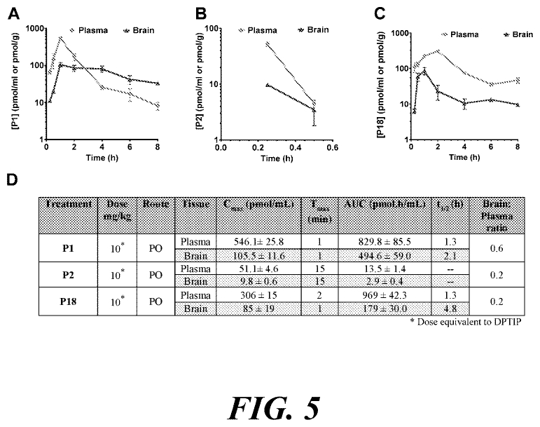

As described earlier, we identified P1 and P18 as the most promising candidates for time dependent PK. Detailed PK parameters of P1, P18 and DPTIP are given in the FIG. 2D and FIG. 5. Following oral administration, P1 exhibited 3.6-fold higher exposure in brain (AUC₀₋ₜ=187.1 vs. 52.8 pmol.h/g) and a 3.7-fold improvement in brain penetration index (AUC_{brain/plasma} ratio 0.70 vs. 0.19) with a substantial improvement in apparent half-life (t_{1/2}˜3 h vs. <30 m) when compared to equimolar DPTIP (FIG. 2A). P1 also demonstrated good plasma and brain exposures for the intact compound (AUC₀₋ₜ=829.9 pmol.h/mL and AUC₀₋ₜ=494.6 pmol.h/g, respectively) depicting about 40% conversion of P1 to DPTIP. P18 which showed best profile in single time point PK exhibited a remarkable 4.7-fold higher exposure in brain (AUC₀₋ₜ=247 vs. 52.8 pmol.h/g), and a 4-fold higher exposure in plasma (AUC0-t=1047 vs. 270 pmol.h/mL), with a substantially improved apparent half-life (t_{1/2}˜2 h vs. <30 m) when compared to equimolar DPTIP. P18 also showed a 1.3-fold improvement in brain penetration index (AUC_{brain/plasma} ratio 0.24 vs. 0.19). Intact P18 showed good plasma and brain exposures (AUC₀₋ₜ=969 pmol.h/mL and AUC₀₋ₜ=179 pmol.h/g respectively) with >50% conversion to DPTIP in the brain in vivo. Overall, since P18 demonstrated best pharmacokinetic profile it was selected for assessment in a mouse model of brain injury for inhibition of EV release as well as target engagement measured by inhibition of nSMase2 activity.

### 2.4.6 P18 Inhibited EV Release and Showed Target Engagement in a Mouse Model of Brain Injury

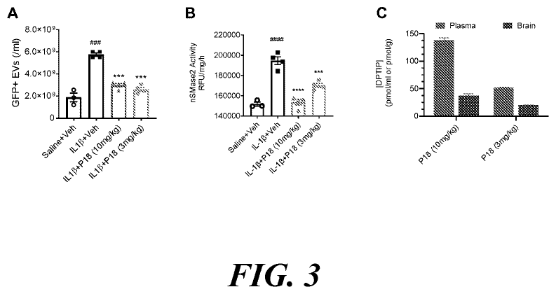

We have previously reported that nSMase2 regulates the secretion of extracellular vesicles from astrocytes in response to a focal inflammatory brain lesion. Rojas et al., 2018. Striatal injection of IL-1β in mice expressing GFAP-EGFP in astroglia evokes the release of GFP-labelled EVs that rapidly enter into plasma and target multiple organ systems. Dickens et al., 2017. We used this mouse model to evaluate the ability of P18 to inhibit EV release by the inhibition of nSMase2 activity in vivo. The PK results (FIG. 2) showed that the released DPTIP concentrations from P18 (10 mg/kg PO) were above the IC₅₀ (30 nM) of DPTIP until at-least 4 hours and also provided approximately 4-fold higher exposures compared to equimolar DPTIP. Therefore, we used both 3 and 10 mg/kg (DPTIP equivalent) PO to evaluate its dose-response effects. Interleukin-1β-injected mice, orally pre-dosed with P18 (30 min prior) were euthanized 4-hours post-IL-1β injection. GFP labeled (GFP+) EVs were quantified in plasma and nSMase2 activity was measured in the brain striata. DPTIP levels were also measured in mouse plasma and brain to confirm brain penetration in this mouse model. Intrastriatal administration of IL-1β significantly increased the number of GFP+ EVs in plasma, and P18 inhibited the release of brain derived GFP+ EVs into blood at both 3 and 10 mg/kg (DPTIP equivalent dose) (FIG. 3A). Similarly, P18 treatment reduced nSMase2 activity increased in response to IL-1β in a dose dependent manner (FIG. 3B). The levels of released DPTIP and PK parameters in plasma and brain samples from IL-1β treated mice were similar in normal mice and mice administered intrastriatal IL-1β.

### 2.5 Summary

Cumulative evidence suggests that EVs play a key role in the pathophysiology of various diseases including neurological disorders, cancer and HIV (for review see Tallon et al., 2021). Development of small molecule inhibitors of nSMase2 would enable us to further investigate the pathogenic role played by EVs as well as the therapeutic utility of blocking nSMase2-mediated production and release of EVs pharmacologically. DPTIP represents the most potent nSMase2 inhibitor identified to date. However, DPTIP exhibits poor PK in mice with low exposures in plasma and brain. Our previous SAR studies on the DPTIP scaffold revealed key pharmacophores essential for potent inhibition as well as structural modifications that can be tolerated by the enzyme. However, these structural analogs of DPTIP failed to improve upon PK properties of these nSMase2 inhibitors.

In an effort to address poor PK properties exhibited by DPTIP and enhance its delivery to the brain we employed a prodrug strategy. We strategized to mask the polar hydroxyl group of DPTIP by various promoieties, such as simple alkyl chain esters or carbamates, piperidinyl carbamates, phosphate esters, to improve lipophilicity, oral absorption. and brain penetration. Furthermore, we also strategized to use dipeptide promoieties for active transport via peptide transporters and nicotinic promoiety for improve brain uptake through passive diffusion. We designed and synthesized DPTIP prodrugs P1-P18, and evaluated them using in vitro stability, in vivo PK, EV release inhibition, and target engagement assays. P18 showed excellent improvement of plasma exposures (approximately 4.0-fold) and half-life (2-3 h) over DPTIP. While P1 showed a 3.7-fold improvement in the brain penetration index, P18 demonstrated the highest improvement in brain exposure (4.7-fold) following PO administration in mice as compared to DPTIP. P18 also demonstrated significant inhibition of nSMase2 activity and IL-1β-induced EV release in mice. Using a prodrug approach, we achieved a significant enhancement in the oral availability, brain penetration, and apparent half-life of DPTIP and these outcomes could aid in its clinical translation.

### 2.6 Experimental

### 2.6.1 General Synthetic Procedure for Prodrugs P1, P7-12:

To a stirred solution of DPTIP (800 mg, 2.11 mmol, 1.0 equiv.) in 1,2-dichloroethane (25 mL) cooled to 0° C. was added DIPEA (1.80 mL, 10.6 mmol, 5.0 equiv.) and triphosgene (692 mg, 2.32 mmol, 1.1 equiv.) and the resulting mixture was stirred for 1 h at 0° C. Primary or secondary amine (3.17 mmol, 1.5 equiv.) was added to the above reaction mixture and the mixture was stirred at room temperature for further 3 h. The completion of the reaction was monitored by TLC. The reaction mixture was diluted with DCM (50 mL), washed with water (2×25 mL) and brine solution (25 mL). Organic phase was separated, dried over anhydrous sodium sulphate and solvents were evaporated under reduced pressure. The residue was purified by Biotage isolera or preparative HPLC to give resulting products P1, P7-P12 in 25-40% yields. ¹H NMR was recorded on a Bruker 400 MHz spectrometer, using residual signal of deuterated solvent as internal reference. Chemical shifts (δ) are reported in ppm relative to tetramethylsilane. ¹H NMR data are reported as follows: chemical shift (multiplicity, coupling constants, and number of hydrogens). Multiplicity is abbreviated as follows: s (singlet), d (doublet), t (triplet), q (quartet), m (multiplet), br (broad).

### 2.6.2 Key Compounds Purity

HPLC analysis was performed with a X-Bridge C8 (50×4.6mm) 3.5 μm reversed-phase column for assessing the purity of the prodrugs. The purity for all key compounds was >95%.

The analysis was performed using one of the following gradients: Method A: mobile phase A: 0.1% trifluoracetic acid in water; mobile phase B: acetonitrile; flow: 2.0 mL/min. Method B: mobile phase A: 0.1% trifluoracetic acid in water; mobile phase B: acetonitrile; flow: 1.0 mL/min. Method C: mobile phase A: 10 mM NH4HCO3 in water; mobile phase B: acetonitrile; flow: 1.0 mL/min. Method D: mobile phase A: 0.1% trifluoracetic acid in water; mobile phase B: 0.1% trifluoracetic acid in acetonitrile; flow: 2.0 mL/min. Method E: column: Greensep Silica (250×4.6 mm), 5μ; mobile phase A: n-hexane; mobile phase B: ethanol; flow: 1.0 mL/min. Method F: mobile phase A: 0.1% Formic acid in water; mobile phase B: acetonitrile; flow: 2.0 mL/min. Description of the purity analysis has been included in Section 2.6.

### 2.6.3 Representative Compounds

2,6-Dimethoxy-4-(4-phenyl-5-(thiophen-2-yl)-1H-imidazol-2-yl)phenyl[1,4′-bipiperidine]-1′-carboxylate (P1): 1,4′-bipiperidine (533 mg): mobile phase: 5-10% MeOH in DCM; P1 was obtained as an off white solid (483 mg) in 40% yield. ¹H-NMR (400 MHz, DMSO-d₆): δ 12.77 (s, 1H), 8.17 (t, J=8.0 Hz, 2H), 7.61 (t, J=8.0 Hz, 1H), 7.54-7.32 (m, 5H), 7.06 (s, 1H), 6.98 (s, 1H), 4.17-4.02 (m, 1H), 4.01-3.98 (m, 1H), 3.84 (s, 6H), 2.86-2.68 (m, 2H), 2.51-2.50 (m, 4H), 2.33-2.17 (m, 2H), 1.76-1.40 (m, 9H). HRMS (ESI): [M+H]⁺ (C₃₂H₃₇N₄O₄S) calculated 573.2535, found 573.2527.
2,6-Dimethoxy-4-(5-phenyl-4-(thiophen-2-yl)-1H-imidazol-2-yl)phenyl methyl (pentyl) carbamate (P7): N-methylpentan-1-amine (321 mg); preparative HPLC; P7 was obtained as an off white solid (277 mg) in 26% yield. ¹H-NMR (400 MHz, DMSO-d₆): δ 12.77 (s, 1H), 9.93 (d, J=8.0 Hz, 2H), 7.62-7.30 (m, 6H), 7.06-6.98 (m, 2H), 3.91 (s, 6H), 3.40-3.38 (m, 2H), 3.28-3.26 (m, 1H), 3.02 (s, 3H), 1.65-1.52 (m, 1H), 1.33-1.17 (m, 4H), 0.91-0.86 (m, 3H), HRMS (ESI): [M+H]⁺ (C₂₈H₃₂N₃O₄S) calculated 506.2113, found 506.2114.
2,6-Dimethoxy-4-(5-phenyl-4-(thiophen-2-yl)-1H-imidazol-2-yl)phenyl pentylcarbamate (P8): pentan-1-amine (276 mg); prep-HPLC; P8 was obtained as an off white solid (270 mg) in 26% yield. ¹H-NMR (400 MHz, DMSO-d₆): δ 12.75 (s, 1H), 7.64-7.60 (m, 3H), 7.54-7.38 (m, 5H), 7.37-7.34 (m, 1H), 7.06-6.97 (m, 2H), 3.84 (s, 6H), 3.04 (t, J=4.0 Hz, 2H), 1.47-1.45 (m, 2H), 1.31-1.29 (m, 4H), 0.90 (t, J=12.0 Hz, 3H). HRMS (ESI): [M+H]⁺ (C₂₇H₃₀N₃O₄S) calculated 492.1957, found 492.1960.
2,6-Dimethoxy-4-(5-phenyl-4-(thiophen-2-yl)-1H-imidazol-2-yl)phenyl heptylcarbamate (P9): heptan-1-amine (365 mg); preparative HPLC; P9 was obtained as an off white solid (296 mg) in 27% yield. ¹H-NMR (400 MHz, DMSO-d₆): δ 12.75 (s, 1H), 7.65-7.34 (m, 9H), 7.05-6.98 (m, 2H), 3.85 (s, 6H), 3.06-3.02 (m, 2H), 1.47-1.44 (m, 2H), 1.29-1.24 (m, 8H), 0.89 (t, J=8.0 Hz, 3H). HRMS (ESI): [M+H]⁺ (C₂₉H₃₄N₃O₄S) calculated 520.2270, found 520.2277.
2,6-Dimethoxy-4-(5-phenyl-4-(thiophen-2-yl)-1H-imidazol-2-yl)phenyl 2,6-diethylpiperidine-1-carboxylate (P10): 2,6-diethylpiperidine (448 mg); preparative HPLC; P10 was obtained as a pale brown solid (288 mg) in 25% yield. ¹H-NMR (400 MHz, DMSO-d₆): δ 12.76 (s, 1H), 7.63-7.60 (m, 2H), 7.54-7.34 (m, 6H), 7.06-6.97 (m, 2H), 4.03-4.02 (m, 1H), 3.86 (s, 6H), 2.68-2.67 (m, 3H), 1.70-1.54 (m, 8H), 0.90 (t, J=8.0 Hz, 6H), HRMS (ESI): [M+H]⁺ (C₃₁H₃₆N₃O₄S) calculated 546.2426, found 546.2432.
2,6-Dimethoxy-4-(5-phenyl-4-(thiophen-2-yl)-1H-imidazol-2-yl)phenyl dipropylcarbamate (P11): diisopropylamine (321 mg); preparative HPLC; P11 was obtained as an off white solid (288 mg) in 27% yield. ¹H-NMR (400 MHz, DMSO-d₆): δ 12.80 (s, 1H), 7.61 (d, J=8.0 Hz, 2H), 7.48-7.12 (m, 6H), 7.07-6.99 (m, 2H), 3.84 (s, 6H), 3.22-3.21 (m, 2H), 3.19-3.17 (m, 2H), 1.70-1.53 (m, 4H), 0.92 (t, J=8.0 Hz, 6H). HRMS (ESI): [M+H]⁺ (C₂₈H₃₂N₃O₄S) calculated 506.2113, found 506.2118.
2,6-Dimethoxy-4-(5-phenyl-4-(thiophen-2-yl)-1H-imidazol-2-yl)phenyl 8-azabicyclo[3.2.1]octane-8-carboxylate (P12): 8-azabicyclo[3.2.1]octane hydrochloride (468 mg); preparative HPLC; P12 was obtained as an off white solid (272 mg) in 25% yield. ¹H-NMR (400 MHz, DMSO-d₆): δ 12.76 (s, 1H), 7.61 (d, J=8.0 Hz, 2H), 7.54-7.51 (m, 2H), 7.47-7.32 (m, 4H), 7.06-6.97 (m, 2H), 4.30-4.28 (m, 1H), 4.09-4.03 (m, 1H), 3.87 (s, 6H), 1.99-1.80 (m, 3H), 1.77-1.73 (m, 4H), 1.60-1.43 (m, 3H). HRMS (ESI): [M+H]⁺ (C₂₉H₃₀N₃O₄S) calculated 516.1957, found 516.1961.
Synthesis of 2,6-diethylpiperidine: To a stirred solution of 2,6-diethylpyridine (1.50 g, 11.1 mmol, 1.0 equiv.) in methanol (25 mL) was added 5% rhodium on carbon (300 mg) and few drops of acetic acid. The mixture was stirred at 60° C.for 16 h in an autoclave at 20 atm hydrogen pressure. The completion of the reaction was monitored by TLC. The reaction mixture was filtered through pad of celite and methanol was evaporated under reduced pressure to give 2,6-diethylpiperidine as a colorless liquid (830 mg) in 53% yield.

### 2.6.4 General Synthetic Procedure for Prodrugs P2-P5

To a stirred solution of DPTIP (200 mg, 0.528 mmol, 1.0 equiv.) in 1,2-dichloroethane (5 mL) was added DIPEA (P2: 0.46 mL, 2.64 mmol, 5.0 equiv.; P3-P5: 0.27 mL, 1.58 mmol, 3.0 equiv.) and acyl chloride (1.06 mmol, 2.0 equiv.) at room temperature. The mixture was stirred at room temperature for 8 h (P2). 12 h (P3-P4) or 20 h (P5). The completion of the reaction was monitored by TLC. The reaction mixture was diluted with DCM (10 mL), washed with water (2×10 mL) and brine solution (10) mL). Organic layer was separated, dried over anhydrous sodium sulphate and solvents were evaporated under reduced pressure. The residue was purified by Biotage isolera using 60-70% (P2) or 70-80% (P3-P5) ethyl acetate in petroleum ether as eluents to give off white solids P2-P5 in 15-60% yields.

2,6-Dimethoxy-4-(4-phenyl-5-(thiophen-2-yl)-1H-imidazol-2-yl)phenyl hexanoate (P2): hexanoyl chloride (142 mg): P2 (93.1 mg), 37% yield. ¹H-NMR (400 MHz, DMSO-d₆): δ 12.82 (s, 1H), 7.62-7.21 (m, 8H), 7.06 (s, 1H), 6.99 (s, 1H), 3.85 (s, 6H), 3.32-3.26 (m, 2H), 1.68-1.62 (m, 2H), 1.37-1.32 (m, 4H), 0.92 (t, J=8.0 Hz, 3H). HRMS (ESI): [M+H]⁺ (C₂₇H₂₉N₂O₄S) calculated 477.1848, found 477.1841.
2,6-Dimethoxy-4-(4-phenyl-5-(thiophen-2-yl)-1H-imidazol-2-yl)phenyl acetate (P3): acetyl chloride (83.4 mg); P3 (55.5 mg), 25% yield. ¹H-NMR (400 MHz, DMSO-d₆): δ 12.79 (s, 1H), 7.62-7.34 (m, 8H), 7.06 (s, 1H), 6.98 (s, 1H), 3.86 (s, 6H), 2.28 (s, 3H). HRMS (ESI): [M+H]⁺ (C₂₃H₂₁N₂O₄S) calculated 421.1222, found 421.1223.
2,6-Dimethoxy-4-(4-phenyl-5-(thiophen-2-yl)-1H-imidazol-2-yl)phenyl pivalate (P4): pivaloyl chloride (127 mg); P4 (147 mg), 60% yield. ¹H-NMR (400 MHz, DMSO-d₆): δ 12.82 (s, 1H), 7.68-7.20 (m, 8H), 7.06 (s, 1H), 6.98 (s, 1H), 3.84 (s, 6H), 1.32 (s, 9H). HRMS (ESI): [M+H]⁺ (C₂₆H₂₇N₂O₄S) calculated 463.1691, found 463.1691.
2,6-Dimethoxy-4-(4-phenyl-5-(thiophen-2-yl)-1H-imidazol-2-yl)phenyl dimethylcarbamate (P5): dimethylcarbamic chloride (114 mg). P5 (35.6 mg), 15% yield. ¹H-NMR (400 MHz, DMSO-d₆): δ 12.76 (s, 1H), 7.67-7.60 (m, 2H), 7.56-7.24 (m, 6H), 6.99 (s, 1H), 6.97 (s, 1H), 3.85 (s, 6H), 3.05 (s, 3H), 2.90 (s, 3H). HRMS (ESI): [M+H]⁺ (C₂₄H₂₄N₃O₄S) calculated 450.1487, found 450.1488.

### 2.6.5 Synthesis of Prodrug P6

Chloro(phenyl)methyl (2,6-dimethoxy-4-(4-phenyl-5-(thiophen-2-yl)-1H-imidazol-2-yl)phenyl) carbonate (1): To a stirred solution of DPTIP (400 mg, 1.06 mmol, 1.0 equiv.) in 1,2-dichloroethane (15 mL) cooled to 0° C. was added DIPEA (0.92 mL, 5.28 mmol, 5.0 equiv.) and chloro(phenyl)methyl carbonochloridate (325 mg, 1.59 mmol, 1.5 equiv.). The resulting mixture was stirred at room temperature for 8 h. The completion of the reaction was monitored by LC-MS. Then the reaction mixture was diluted with DCM (20) mL), washed with water (2×20 mL) and brine solution (20 mL). Organic layer was separated, dried over anhydrous sodium sulphate and solvents were evaporated under reduced pressure. The crude product 1 (320 mg) was used in the subsequent step without further purification.
(((2,6-Dimethoxy-4-(4-phenyl-5-(thiophen-2-yl)-1H-imidazol-2-yl)phenoxy)carbonyl)oxy)(phenyl)methyl pivalate (P6): To a stirred solution of intermediate 1 (320 mg, 0.584 mmol, 1.0 equiv.) in acetone (10 mL) was added triethylamine (0.41 mL, 2.92 mmol, 5.0 equiv.) and pivalic acid (179 mg, 1.75 mmol, 3.0 equiv.) at room temperature. The mixture was stirred at room temperature for 48 h. The completion of the reaction was monitored by TLC. The reaction mixture was diluted with ethyl acetate (20 mL), washed with water (2×10 mL) and brine solution (10 mL). Organic layer was separated, dried over anhydrous sodium sulphate and solvents were evaporated under reduced pressure. The residue was purified by reverse phase prep-HPLC to give P6 as an off white solid (35.8 mg) in 10% yield. ¹H-NMR (400 MHz, DMSO-d₆): δ 12.82 (s, 1H), 7.63-7.60 (m, 4H), 7.58-7.51 (m, 5H), 7.47-7.44 (m, 3H), 7.39-7.34 (m, 2H), 7.06 (s, 1H), 6.97 (s, 1H), 3.86 (s, 6H), 1.24 (s, 9H). HRMS (ESI): [M+H]⁺ (C₃₄H₃₃N₂O₇S) calculated 613.2008, found 613.1998.

### 2.6.6 Synthesis of Prodrug P13

tert-Butyl 3-(piperidin-1-yl)-8-azabicyclo[3.2.1]octane-8-carboxylate: To a stirred solution of tert-butyl 3-oxo-8-azabicyclo[3.2.1]octane-8-carboxylate (2.00 g, 8.87 mmol, 1.0 equiv.) in methanol (20 mL) was added piperidine (1.30 g, 13.3 mmol, 1.5 equiv.), few drops of acetic acid and sodium cyanoborohydride (836 mg, 13.3 mmol, 1.5 equiv.) at 0° C. The mixture was stirred at room temperature for 18 h. The completion of the reaction was monitored by TLC. The reaction mixture was diluted with DCM (50 mL), washed with water (25 mL) and brine solution (20 mL). Organic phase was separated, dried over anhydrous sodium sulphate and solvents were evaporated under reduced pressure. The residue was purified by silica gel column chromatography to give tert-butyl 3-(piperidin-1-yl)-8-azabicyclo[3.2.1]octane-8-carboxylate as a colorless liquid (2.09 g) in 80% yield.
3-(Piperidin-1-yl)-8-azabicyclo[3.2.1]octane hydrochloride (Slatter et al., 2000): To a stirred solution of tert-butyl 3-(piperidin-1-yl)-8-azabicyclo[3.2.1]octane-8-carboxylate (1.20 g, 5.08 mmol, 1.0 equiv.) in 1,4-dioxane (10 mL) was added 4M solution of HCl in 1,4-dioxane (6 mL) at 0° C. The mixture was stirred at room temperature for 5 h. The completion of the reaction was monitored by TLC. The volatiles were removed under reduced pressure to give 3-(piperidin-1-yl)-8-azabicyclo[3.2.1]octane hydrochloride as a colorless liquid (879 mg) in 75% yield. The crude product was used to the following step without further purification.
2,6-Dimethoxy-4-(5-phenyl-4-(thiophen-2-yl)-1H-imidazol-2-yl)phenyl 3-(piperidin-1-yl)-8-azabicyclo[3.2.1]octane-8-carboxylate (P13): To a stirred solution of DPTIP (800 mg, 2.11 mmol, 1.0 equiv.) in 1,2-dichloroethane (25 mL) was added DIPEA (1.80 mL, 10.6 mmol, 5.0 equiv.) and triphosgene (692 mg, 2.32 mmol, 1.1 equiv.) at 0° C. The mixture was stirred at 0° C.for 1 h. 3-(Piperidin-1-yl)-8-azabicyclo[3.2.1]octane hydrochloride (700 mg, 3.17 mmol, 1.5 equiv.) was added to the above reaction mixture and the mixture was stirred at room temperature for 3 h. The completion of the reaction was monitored by TLC. The reaction mixture was diluted with DCM (50 mL), washed with water (2×25mL) and brine solution (25mL). Organic phase was separated, dried over anhydrous sodium sulphate and volatiles were removed under reduced pressure. The residue was purified by prep-HPLC to give P13 as an off white solid (316 mg) in 25% yield. ¹H-NMR (400 MHz, DMSO-d₆): δ 12.83 (s, 1H), 7.68-7.60 (m, 4H), 7.55-7.40 (m, 4H), 7.06-6.97 (m, 2H), 4.35-4.33 (m, 1H), 4.14-4.12 (m, 1H), 3.84 (s, 6H), 2.97-2.96 (m, 1H), 2.67-2.51 (m, 4H), 2.01-1.94 (m, 3H), 1.75-1.72 (m, 5H), 1.69-1.63 (m, 4H), 1.41-1.40 (m, 2H). HRMS (ESI): [M+H]⁺ (C₃₄H₃₉N₄O₄S) calcd 599.2692, found 599.2697.

### 2.6.7 Synthesis of Prodrug P14

2,6-Dimethoxy-4-(4-phenyl-5-(thiophen-2-yl)-1H-imidazol-2-yl)phenyl dihydrogen phosphate (P14): To a stirred solution of DPTIP (200 mg, 0.528 mmol, 1.0 equiv.) in 1,2-dichloroethane (5 mL) was added DIPEA (0.46 mL, 2.64 mmol, 5.0 equiv.) and POCl₃ (122 mg, 0.793 mmol, 1.5 equiv.) at 0° C. The mixture was stirred at 0° C. for 2 h. To the resulting mixture was added potassium carbonate (365 mg, 2.64 mmol, 5.0 equiv.) and water (5 mL) and the mixture was stirred at room temperature for further 18 h. The completion of the reaction was monitored by TLC. The reaction mixture was completely concentrated under reduced pressure. The residue was purified by preparative HPLC to give P14 as an off white solid (53.3 mg) in 22% yield. ¹H-NMR (400 MHz, DMSO-d₆): δ 13.39 (s, 1H), 7.63 (t, J=4.0 Hz, 2H), 7.57-7.44 (m, 6H), 7.28 (s, 1H), 7.17 (s, 1H), 3.86 (s, 6H), HRMS (ESI): [M+H]⁺ (C₂₁H₂₀N₂O₆PS) calculated 459.0779, found 459.0780.

### 2.6.8 Synthesis of Prodrug P15

Di-tert-butyl ((2,6-dimethoxy-4-(4-phenyl-5-(thiophen-2-yl)-1H-imidazol-2-yl)phenoxy)methyl) phosphate (4): To a stirred solution of DPTIP (1.50 g, 3.96 mmol, 1.0 equiv.) in DMF (20 mL) was added cesium carbonate (2.58 g, 7.92 mmol, 2.0 equiv), di-tert-butyl (chloromethyl) phosphate (2.05 g, 7.92 mmol, 2.0 equiv) and sodium iodide (594 mg, 3.96 mmol, 1.0 equiv.) at room temperature. The mixture was stirred at room temperature for 24 h. The completion of the reaction was monitored by TLC. The reaction mixture was diluted with ethyl acetate (50 mL), washed with water (2×25 mL) and brine solution (25 mL). Organic layer was separated, dried over anhydrous sodium sulphate and volatiles were evaporated under reduced pressure. The residue was purified by biotage isolera using 60-70% ethyl acetate in petroleum ether as eluent to give intermediate 4 as a pale-yellow solid (1.14 g) in 48% yield.
(2,6-Dimethoxy-4-(4-phenyl-5-(thiophen-2-yl)-1H-imidazol-2-yl)phenoxy) methyl dihydrogen phosphate (P15): To a stirred solution of intermediate 4 (200 mg, 0.333 mmol, 1.0 equiv.) in DCM (5 mL) was dropwise added trifluoroacetic acid (2 mL) in DCM (18 mL). The mixture was stirred at room temperature for 12 h. The completion of the reaction was monitored by TLC. The volatiles were evaporated under reduced pressure. The residue was purified by reverse phase prep-HPLC to give P15 as off white solid (48.8 mg) in 30% yield. ¹H-NMR (400 MHz, DMSO-d₆): δ 7.62 (t, J=4.0 Hz, 2H), 7.45-7.03 (m, 8H), 5.31 (s, 2H), 3.86 (s, 6H). HRMS (ESI): [M+H]⁺ (C₂₂H₂₂N₂O₇PS) calculated 489.0885, found 489.0877.

### 2.6.9 Synthesis of Prodrug P16

tert-Butyl ((2,6-dimethoxy-4-(4-phenyl-5-(thiophen-2-yl)-1H-imidazol-2-yl)phenoxy)methyl) hydrogen phosphate (5): To a stirred solution of intermediate 4 (520 mg, 0.866 mmol, 1.0 equiv.) in methanol (10 mL) was added silica gel (5 g, mess size 60-120 μm) and few drops of HCl. The mixture was stirred at room temperature for 24 h. The completion of the reaction was monitored by LC-MS. The reaction mixture was filtered and the volatiles were removed under reduced pressure. The crude intermediate 5 (470) mg, quantitative yield) was used to the following step without further purification.
((tert-Butoxy((2,6-dimethoxy-4-(4-phenyl-5-(thiophen-2-yl)-1H-imidazol-2-yl)phenoxy)methoxy)phosphoryl)oxy)methyl isopropyl carbonate: To a stirred solution of intermediate 5 (470 mg, 0.866 mmol, 1.0 equiv.) in DMF (10 mL) was added cesium carbonate (564 mg, 1.73 mmol, 2.0 equiv.), chloromethyl isopropyl carbonate (264 mg, 1.73 mmol, 2.0 equiv.) and sodium iodide (130 mg, 0.866 mmol, 1.0 equiv.) at room temperature. The mixture was stirred at room temperature for 24 h. The completion of reaction was monitored by LC-MS. The reaction mixture was filtered and volatiles were removed under reduced pressure. The crude product was obtained in 48% yield (275 mg) and was used in the subsequent step without further purification.
((((2,6-Dimethoxy-4-(4-phenyl-5-(thiophen-2-yl)-1H-imidazol-2-yl)phenoxy)methoxy)(hydroxy)phosphoryl)oxy)methyl isopropyl carbonate (P16): To a stirred solution of ((tert-butoxy((2,6-dimethoxy-4-(4-phenyl-5-(thiophen-2-yl)-1H-imidazol-2-yl)phenoxy)methoxy)phosphoryl)oxy)methyl isopropyl carbonate (320 mg. 0.484 mmol, 1.0 equiv.) in DCM (5 mL) was slowly added trifluoroacetic acid (2 mL) in DCM (18 mL). The mixture was stirred at room temperature for 12 h. The completion of reaction was monitored by LC-MS. Volatiles were removed under reduced pressure and the residue was purified by reverse phase preparative HPLC to give P16 as an off white solid (32.2 mg) in 11% yield. ¹H-NMR (400 MHz, DMSO-d₆): δ 7.61 (t, J=8.0 Hz, 2H), 7.48 (t, J=8.0 Hz, 2H), 7.41-7.28 (m, 3H), 7.18-7.13 (m, 1H), 7.05 (s, 1H), 7.00 (s, 1H), 5.37-5.31 (m, 4H), 4.81-4.75 (m, 1H), 3.85 (s, 6H), 1.23 (d, J=4.0 Hz, 6H). HRMS (ESI): [M+H]⁺ (C₂₇H₃₀N₂O₁₀PS) calculated 605.1358, found 605.1348.

### 2.6.10 Synthesis of Prodrug P17

2,6-Dimethoxy-4-(4-phenyl-5-(thiophen-2-yl)-1H-imidazol-2-yl)phenyl nicotinate: To the stirred solution of DPTIP (1.00 g, 2.64 mmol, 1.0 equiv.) in DMF (15 mL) was added nicotinic acid (488 mg, 3.96 mmol, 1.5 equiv.), EDC*HCl (760 mg, 3.96 mmol, 1.5 equiv.), HOBt (536 mg, 3.96 mmol, 1.5 equiv.) and triethyalmine (1.10 mL, 7.92 mmol, 3.0 equiv.). The mixture was stirred at room temperature for 12 h until the completion of reaction (monitored by TLC). The reaction mixture was diluted with ethyl acetate (25 mL), washed with water (2×10 mL) and brine solution (10 mL). Organic layer was separated, dried over anhydrous sodium sulphate and solvents were evaporated under reduced pressure. The residue was purified by biotage isolera using 80% ethyl acetate in petroleum ether as an eluent to give 2,6-dimethoxy-4-(4-phenyl-5-(thiophen-2-yl)-1H-imidazol-2-yl) phenyl nicotinate as an off white solid (894 mg) in 70% yield.
3-((2,6-Dimethoxy-4-(4-phenyl-5-(thiophen-2-yl)-1H-imidazol-2-yl)phenoxy)carbonyl)-1-methylpyridin-1-ium iodide (6): To the stirred solution of 2,6-dimethoxy-4-(4-phenyl-5-(thiophen-2-yl)-1H-imidazol-2-yl)phenyl nicotinate (700 mg, 1.44 mmol, 1.0 equiv.) in acetone (20 mL) was added iodomethane (0.18 mL, 2.89 mmol, 2.0 equiv.). The mixture was stirred at room temperature for 24 h. The completion of the reaction was monitored by TLC. The volatiles were removed under reduced pressure to give the title compound 6 as a pale-yellow solid (802 mg) in 89% yield. The crude product was used to the following step without further purification.
2,6-Dimethoxy-4-(4-phenyl-5-(thiophen-2-yl)-1H-imidazol-2-yl)phenyl 1-methyl-1,4-dihydropyridine-3-carboxylate (P17): To a stirred solution of intermediate 6 (780 mg, 1.56 mmol, 1.0 equiv.) in water (20 mL) at 0° C. was added sodium bicarbonate (657 mg, 7.82 mmol, 5.0 equiv.) and sodium dithionite (817 mg. 4.69 mmol, 3.0 equiv.). The mixture was stirred at 0° C. for 3 h. The reaction mixture was diluted with ethyl acetate (25 mL), washed with water (10 mL) and brine solution (10 mL). Organic layer was separated, dried over anhydrous sodium sulphate and ethyl acetate was evaporated under reduced pressure. The residue was purified by reverse phase prep-HPLC to afford P17 as a pale-yellow solid (39.0 mg) in 5% yield. ¹H-NMR (400 MHz, DMSO-d₆): δ 12.80 (s, 1H), 7.62-7.31 (m, 9H), 7.10 (s, 1H), 7.09 (s, 1H), 5.92 (s, 1H), 4.83-4.79 (m, 1H), 3.87 (s, 6H), 3.32-3.04 (m, 5H). HRMS (ESI): [M+H]⁺ (C₂₈H₂₆N₃O₄S) calculated 500.1644, found 500.1642.

### 2.6.11 Synthesis of Prodrug P18

1-Benzyl-2,6-diethylpiperidin-4-one (7): To a stirred solution of 3-oxopentanedioic acid (10.0 g, 68.4 mmol, 1.0 equiv.) in water (50 mL) was added propionaldehyde (9.8 mL, 137 mmol, 2.0 equiv.) and the resulting mixture was stirred at room temperature for 15 min. The reaction mixture was cooled to 0° C., benzylamine (7.4 mL, 68.4 mmol, 1.0 equiv.) was added and mixture was stirred at room temperature for 48 h. The progress of the reaction was monitored by TLC. The reaction mixture was cooled to room temperature, acidified with 1N HCl up to pH=2, later neutralized with saturated NaHCO₃ solution (to pH=7) and extracted with ethyl acetate (2×200 mL). The combined organic layers were dried over anhydrous Na₂SO₄, filtered and concentrated under reduced pressure. The crude compound was purified by column chromatography eluting with 0-50% ethyl acetate in hexane to afford the intermediate 7 as a brown oil (6.5 g) in 65% yield. ¹H-NMR (400 MHz, DMSO-d₆): δ 7.39 (t, J=8.0 Hz, 2H), 7.34 (t, J=8.0 Hz, 2H), 7.22 (t, J=8.0 Hz, 1H), 3.92 (d, J=12.0 Hz, 1H), 3.66 (d, J=12.0 Hz, 1H), 2.93-2.90 (m, 2H), 2.42-2.41 (m, 2H), 2.11-2.10 (m, 2H), 1.55-1.53 (m, 2H), 1.35-1.33 (m, 2H), 0.85 (t, J=8.0 Hz, 6H).
1′-Benzyl-2′,6′-diethyl-1,4′-bipiperidine: To a stirred solution of intermediate 7 (5.00 g, 15.9 mmol, 1.0 equiv.) in methanol (50 mL) was added piperidine (2.00 g, 23.8 mmol, 1.5 equiv.), acetic acid (catalytic) and sodium cyanoborohydride (1.49 g, 23.8 mmol, 1.5 equiv.) at 0° C. The mixture was stirred at room temperature for 16 h. Completion of the reaction was monitored by TLC. The reaction mixture was diluted with ethyl acetate (2×100 mL) and washed with water (50 mL). The combined organic layers were separated, and dried over anhydrous Na₂SO₄, filtered and concentrated under reduced pressure. The crude compound was purified by column chromatography eluting with 0-80% ethyl acetate in hexane to afford the 1′-benzyl-2′,6′-diethyl-1,4′-bipiperidine as a brown gum (3.1 g) in 62% yield.
2′,6′-Diethyl-1,4′-bipiperidine (8): To a degassed solution of 1′-benzyl-2′,6′-diethyl-1,4′-bipiperidine (3.10 g, 9.86 mmol, 1.0 equiv.) in methanol (40 mL) was added 10% of palladium on carbon (600 mg, 0.564 mmol, 0.06 equiv.) and few drops of acetic acid. The mixture was stirred in an autoclave under hydrogen pressure (5 atm) at 50° C. for 16 h. Completion of the reaction was monitored by TLC. The reaction mixture was filtered through a pad of celite and methanol was evaporated under reduced pressure to give intermediate 8 (1.28 g) in 58% yield.
2,6-Dimethoxy-4-(5-phenyl-4-(thiophen-2-yl)-1H-imidazol-2-yl)phenyl 2′,6′-diethyl-[1,4′-bipiperidine]-1′-carboxylate (P18): To a stirred solution of DPTIP (800 mg, 2.11 mmol, 1.0 equiv.) in 1,2-dichloroethane (25 mL) was added DIPEA (1.80 mL, 10.6 mmol, 5.0 equiv.) and triphosgene (692 mg, 2.32 mmol, 1.1 equiv.) at 0° C. The mixture was stirred at 0° C. for 1 h. 2′,6′-Diethyl-1,4′-bipiperidine 8 (711 mg, 3.17 mmol, 1.5 equiv.) was added to the above reaction mixture and the resulting mixture was stirred at room temperature for 3 h. The completion of the reaction was monitored by TLC. The reaction mixture was diluted with DCM (50 mL), washed with water (2×25mL) and brine solution (25 mL). The organic layer was separated, dried over anhydrous sodium sulphate and volatiles were removed under reduced pressure. The residue was purified by prep-HPLC to give P18 as an off white solid (332 mg) in 25% yield. ¹H-NMR (400 MHz, DMSO-d₆): δ 12.96 (s, 1H), 7.61 (t, J=8.0 Hz, 2H), 7.47-7.41 (m, 8H), 4.04-4.01 (m, 1H), 3.85 (s, 6H), 2.69 (t, J=8.0 Hz, 1H), 2.51-2.50 (m, 3H), 1.96-1.92 (m, 5H), 1.75-1.73 (m, 2H), 1.53-1.50 (m, 7H), 1.40-1.39 (m, 2H), 0.92 (t, J=8.0 Hz, 6H). HRMS (ESI): [M+H]⁺ (C₃₆H₄₅N₄O₄S) calculated 629.3161, found 629.3149.

### 2.6.12 Metabolic Stability Studies

The in vitro metabolic stability analysis of DPTIP prodrugs were performed in mouse plasma, liver, and brain homogenates as previously described. Zimmermann et al., 2018. For the tissue homogenates, washed tissues were diluted 10-fold in 0.1 M potassium phosphate buffer and homogenized using a probe sonicator. To evaluate the stability of the intact prodrug over time, each of the crude homogenates and plasma were aliquoted to 1 mL and then spiked with a final assay concentration of 10 μM of each prodrug followed by incubation in an orbital shaker at 37° C. for 1 h (in triplicate). Sample from each incubation at predetermined time points (0 min, and 1 h) was quenched with three volumes of acetonitrile containing the internal standard (IS; losartan: 0.5 μm). Samples were vortex-mixed for 30 secs and centrifuged at 10,000×g for 10 min at 4° C. Disappearance of intact prodrugs from these samples were performed on a Dionex ultra-high-performance LC system coupled with Q Exactive Focus orbitrap mass spectrometer (Thermo Fisher Scientific Inc., Waltham MA). The separation of analytes was achieved using the Agilent Eclipse Plus column (100×2.1 mm i.d.; maintained at 35° C.) packed with a 1.8 μm C18 stationary phase. The mobile phase consisted of 0.1% formic acid in water and 0.1% formic acid in acetonitrile. Pumps were operated at a flow rate of 0.4 mL/min for 9 min using gradient elution. The mass spectrometer controlled by Xcalibur software 4.0.27.13 (Thermo Scientific) was operated with a heated electrospray ionization (HESI) ion source in positive ionization mode. Quantification of the prodrugs were performed in the full-scan mode (from m/z 50 to 1600) by comparing t=0 samples with t=60 min samples.

### 2.6.13 Pharmacokinetic Study in Mice

All PK studies in mice were conducted according to protocols approved by the Animal Care and Use Committee at Johns Hopkins University. Male CD-1 mice between 25 and 30 g were obtained from Harlan and maintained on a 12 h light-dark cycle with ad libitum access to food and water. DPTIP was administered orally (PO) at a dose of 10 mg/kg (5% DMSO, 10% Tween-80 in saline). Prodrugs were dose at a dose of 10 mg/kg DPTIP equivalent (5% DMSO, 10% Tween-80 in saline) and administered as a single peroral (PO) dose. All of the formulations were freshly prepared prior to the dosing. The mice were sacrificed at specified time points (either 2 h or 0.25, 0.5, 1, 2, 4, 6, and 8 h) post drug administration. For the collection of plasma and brain tissue, animals were euthanized with CO₂, and blood samples were collected in heparinized microtubes by cardiac puncture. Brains were dissected and immediately flash frozen (−80° C.) Blood samples were spun at 2000 g for 15 min, and plasma was removed and stored at −80° C. until LC-MS/MS analysis. Prior to extraction, frozen samples were thawed on ice.

### 2.6.14 Bioanalysis

Quantitation of the prodrugs and the DPTIP released from prodrugs were performed using our published DPTIP bioanalytical LC/MS/MS method, Rojas et al., 2018: Rojas et al., 2019, with necessary minor modifications. Briefly, calibration standards were prepared using respective tissue (naïve plasma and brain) with additions of respective DPTIP prodrugs and DPTIP. For quantifying the intact prodrugs and released DPTIP in the PK samples, plasma samples (20 μL) were processed using a single liquid extraction method by addition of 100 μL of acetonitrile containing internal standard (losartan: 0.5 μM), followed by vortex-mixing for 30 s and then centrifugation at 10,000×g for 10 min at 4° C. Brain tissues were diluted 1:5 w/v with acetonitrile containing losartan (0.5 μm) and homogenized, followed by vortex-mixing and centrifugation at 10,000 g for 10 min at 4° C. A 50 μL aliquot of the supernatant were diluted with 50 uL of water and transferred to 250 μL polypropylene autosampler vials sealed with teflon caps. Then, 2 μL of the sample were injected into the LC/MS/MS system for analysis. Chromatographic analysis was performed using an Accela ultra-high-performance system consisting of an analytical pump and an autosampler coupled with a TSQ Vantage mass spectrometer. Separation of analyte was achieved at ambient temperature using Agilent Eclipse Plus column (100×2.1 mm i.d.) packed with a 1.8 μm C18 stationary phase. The mobile phase consisted of 0.1% formic acid in acetonitrile and 0.1% formic acid in water with gradient elution were used. The [M+H]⁺ ion transition of DPTIP (m/z 378.956→363.073, 200.055) and losartan (IS) (m/z 423.200→207.107, 180.880) were used. Plasma concentrations (nmol/ml) as well as brain tissue concentrations (nmol/g) were determined and plots of mean plasma concentration versus time were constructed. Non-compartmental analysis modules in Phoenix WinNonlin version 7.0 (Certara USA, Inc., Princeton, NJ) were used to quantify exposures (AUC₀₋ₜ).

### 2.6.15 Measurement of EV Release In Vivo in Mice

Striatal injections and EV measurements were performed as previously described by our group. Dickens et al., 2017; Mccluskey et al., 2008. Briefly, male (2-3 month), GFAP-EGFP transgenic mice that allow for the evaluation of fluorescent EVs in plasma that are generated in the brain (Jackson Laboratories, n=4) were anaesthetized with 3% isoflurane (Baxter) in oxygen (Airgas) and placed in a stereotaxic frame (Stoelting Co). We chose male mice to avoid effect of female specific hormones (i.e., estrogen) in inflammation that is known to be anti-inflammatory. Zhang et al., 2014. A small burr hole was drilled in the skull over the left striatum using a dental drill (Fine Scientific Tools). IL-1β (0.1 ng/3 μL) was injected (total volume of 3 μL) at the rate 0.5 μL·min−1 via a pulled glass capillary (tip diameter <50 μm). Mccluskey et al., 2008. The stereotaxic coordinates based on bregma as reference point were A/P+1; M/L−2; and −3 D/V. Dickens et al., 2017; Paxinos and Frankline, 2001. Saline was used as a control. P18 (3 and 10 mg/kg DPTIP equivalent, 5% DMSO, 10% Tween-80 in saline) was given orally 30 min before IL-1β injection. Mice subjected to intrastriatal injection of IL-1β were also injected with the NSAID carprofen (rimadyl, 5 mg/kg. i.p.) and closely monitored during recovery; no adverse reactions were observed. Following infusion, the capillary was held in place for 5 min to allow for the solution to diffuse into the tissue. Fifteen mice (n=4 for treatments and vehicle; n=3 for saline+vehicle group) were utilized to perform EVs counts. Mice were euthanized at 4 hr post-IL-1β treatment with overdose of anaesthetic (isoflurane). Blood samples were taken at death by cardiac puncture with heparin (Sigma) coated syringes and EDTA tubes (BD). Blood was immediately centrifuged at 2,700×g for 15 min (20° C.) to obtain plasma. Plasma was further centrifuged at 10,000 g for 15 min (4° C.) to generate platelet-free plasma. This procedure removes large particles such as apoptotic bodies. Quantitation of Plasma EVs: Dynabeads M-450) Epoxy (Invitrogen) were coupled with anti-GFP antibody (Thermo Fisher) at a ratio of 200-μg antibody per 4×10⁸ beads. Plasma from GFAP-GFP mouse (50 μL) was incubated with 2×10⁷ anti-GFP antibody-coupled Dynabeads at 4° C. overnight. The beads were washed and placed on a magnet to separate EVs bound to anti-GFP antibody-coupled Dynabeads. The precipitated EVs were eluted using 0.1-M glycine (pH 3.0). The concentration of immunoprecipitated GFP+ EVs was quantified using ZetaView nanoparticle tracking analysis (Particle Metrix) and corresponding ZetaView software (8.05.14.SP7). The instrument was calibrated with 100 nm diameter beads (Thermo Scientific) prior to use. Instrument preacqusition were set to following parameters: temperature 23° C., sensitivity 85, frame rate of 30 frames per second (fps), shutter speed of 100, and a laser pulse duration equal to that of shutter duration. Post-acquisition parameters were set to a minimum brightness of 25, maximum size of 200 pixels, and a minimum size of 10 pixels. For each sample, 1 ml of diluted EVs were injected into the sample-carrier cell and the particle count was measured at five positions, with two cycles of reading per position. The sample-carrier cell was washed with PBS after every sample. The data were collected by an investigator blinded to experimental conditions.

2.6.16 Measurement of nSMase2 Activity In Vivo in Mice p Interleukin-1β-injected mice, orally pre-dosed with either vehicle or P18 (3 and 10 mg/kg DPTIP equivalent, 30min before IL-1β), were euthanized after four hours of IL-1β injection, the striata dissected and analyzed for nSMase2 activity using a modification of previously published protocols. Rojas et al., 2018; Figuera-Losada et al., 2015. Briefly, mice striata were homogenized in ice-cold Tris-HCl buffer (0.1 M, pH 7.5) containing 250 mM sucrose, 10 mM EGTA (Research Products International, Prospect, IL), 100 μM sodium molybdate and protease inhibitors (Cell Signaling, Danvers, MA) using Biomasher II and then sonicated using Kontes' Micro Ultrasonic Cell Disrupter (three pulses of 15 s duration on ice with 30 s between pulses). The resulting lysates were collected for both nSMase2 activity measurements and total protein analysis. nSMase2 activity measurements were initiated upon the addition of Sphingomyelin (SM) and coupling enzymes in the Amplex Red system (25 μl), Rojas et al., 2018; Figuera-Losada et al., 2015, and SM hydrolysis carried out in total reaction volumes of 50 μl in 384-well microplates for 3 h at 37° C. At the end of the reaction period, the relative fluorescence units were measured at Ex 530 nm, Em 590 nm. Finally, total protein measurements were carried as per manufacturer's instructions using BioRad's Detergent Compatible Protein Assay kit and data presented as RFU/mg/h.

### 2.6.17 Abbreviations

EV=Extracellular vesicle; nSMase2=Neutral Sphingomyelinase 2; SMase=Sphingomyelinase; DPTIP=2,6-Dimethoxy-4-(5-phenyl-4-thiophen-2-yl-1H-imidazol-2-yl)-phenol; AUC=Area under the curve; IL-β: Interleukin-1 beta; CES1: Carboxylesterase-1; DIPEA=N,N-Diisopropylethylamine or Hünig's base; TLC=Thin layer chromatography; TEA=Triethylamine; DCE=Dichloroethane; DCM=Dicholoromethane; TFA=Trifluoroacetic acid; EDCI=1-Ethyl-3-(3-dimethylaminopropyl)carbodiimide; HOBt=Hydroxybenzotriazole; PK=Pharmacokinetics; GI=Gastrointestinal; HPLC=High performance liquid chromatography.

### Example 3

### DPTIP Prodrugs Pharmacokinetics Study in Dogs

An overview of a pharmacokinetics study of the presently disclosed DPTIP prodrugs in dogs is provided in Table 3-1.

TABLE 3-1
Study Design for Pharmacokinetics of Representative DPTIP Prodrugs in Dogs

| | | | | | | Blood |
|---|---|---|---|---|---|---|
| Group | Test | Dose | N = | Dose | | Sampling |
| # | Article | Route | (male) | (mg/kg) | Vehicle | Time Points |
| 1 | DPTIP | IV | 1 | 2 | 20% EtOH: 70% | |
| | | | | | PEG400: 10% DPBS | |
| 2 | Prodrug-P1 | PO | 2 | 3.1 | 5% NMP, 5% | Pre-dose, 5, |
| 3 | Prodrug-P18 | PO | 2 | 3.34 | Kolliphor, 30% | 15, 30, min, |
| | | | | | PEG400, 60% Water | 1, 2, 3, 4, 6, |
| 4 | Prodrug-P15 | PO | 2 | 1.29 | 5% NMP, 5% | 8, and 24 |
| | | | | | Kolliphor, 30% | hours post |
| | | | | | PEG400, 60% Water | dose |
| 5 | Prodrug-P2 | PO | 1 | 1.26 | 5% NMP, 5% | |
| | | | | | Kolliphor, 30% | |
| | | | | | PEG400, 60% Water | |

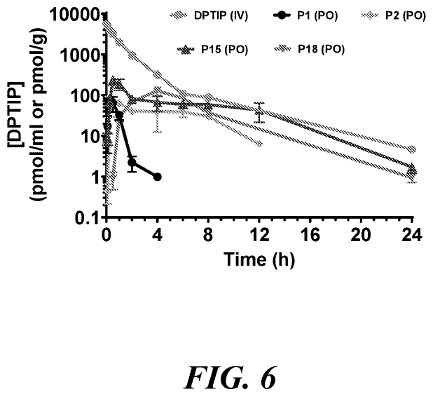

As provided in Table 3-2 and FIG. 6, oral treatment of P1 shows poor oral absorption with <1% oral bioavailability. Prodrug P18 shows moderate oral absorption with approximately 12% oral bioavailability and a decent plasma profile. Prodrug P15 shows a promising oral pharmacokinetic profile with approximately 30% bioavailability. In contrast, prodrug P2 shows poor oral absorption with approximately 12% bioavailability.

TABLE 3-2
Dog Plasma Pharmacokinetics for DPTIP and Prodrugs P1, P2, P15 and P18

| | | | DPTIP | | | |
|---|---|---|---|---|---|---|
| | | Dose | Cₘₐₓ | Tₘₐₓ | DPTIP AUC | Bioavailability |
| Treatment | Route | (mg/kg) | (pmol/mL) | (h) | (pmol · h/mL) | (% F) |
| DPTIP | IV | 2 | 5501 (C₀) | | 7468 | |
| Prodrug-P1 | PO | 3.1 (2 mg/ | 68.4 ± 13.2 | 0.25 | 69.3 ± 12.5 | <1 |
| | | kg eqv) | | | | |
| Prodrug-P18 | PO | 3.34 (2 mg/ | 128.1 ± 20.0 | 4 | 892.8 ± 45.2 | 12 |
| | | kg eqv) | | | | |
| Prodrug-P15 | PO | 1.29 (1 mg/ | 234.3 ± 58.0 | 0.5 | 1149 ± 209 | 30 |
| | | kg eqv) | | | | |
| Prodrug-P2 | PO | 1.26 (1 mg/ | 72.9 ± 7.2 | 0.5 | 447.7 ± 43.4 | 12 |
| | | kg eqv) | | | | |

### Example 4

### Solid Dispersion Formulation With Soluplus® and Labrasol for P18 to Enhance Oral Absorption

P18 was formulated into a solid dispersion formulation with both Soluplus® (polyvinyl caprolactam-polyvinyl acetate-polyethylene glycol graft copolymer (PCL-PVAc-PEG) and Labrasol® (a caprylocaproyl macrogol-8 glyceride). Briefly, 10 mg of P18, and 100 mg of Soluplus® or Labrasol® were dissolved in 5 mL of ethanol by vortexing. The ethanol mixture was then evaporated on a rotary evaporator and stored overnight in a vacuum oven to further dry the contents. The thin film was hydrated with approximately 5 mL of ultrapure water to obtain Soluplus®/Labrasol® micelles containing the prodrugs. The solubility of P18 was below 0.5 mg/mL in water. In contrast, the solubility of the prodrugs, e.g., P18 in the micelle formulation were improved as presented below.

TABLE 4-1
Solid Dispersion of P18 in Soluplus ® or Labrasol ®

| Prodrug | Soluplus ® | Labrasol ® |
|---|---|---|
| P18 | 1.7 mg/mL | 1.7 mg/mL |

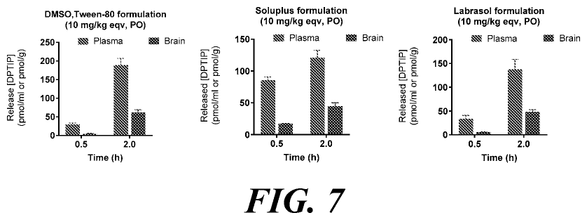

The resulting formulations were analyzed for drug content loading using LC-MS/MS analysis and found to be in the range of 91-97% of label claimed of the prodrug content (for both formulation). We used this formulation and conducted a two-time point (30 min and 2 h) PK in mice and compared the result with the conventional formulation (DMSO, Tween). The conventional formulation was made by dissolving respective prodrug in 5% DMSO, 10% Tween-80 and 85% PBS. As provided in FIG. 7, the P18/Soluplus® formulation showed moderate improvement in plasma and brain DPTIP levels at 30 min time point compared to DMSO-Tween formulation.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents form part of the common general knowledge in the art.
Tallon, C.; Hollinger, K. R.; Pal, A.; Bell, B. J.; Rais, R.; Tsukamoto, T.; Witwer, K. W.; Haughey, N. J.; Slusher, B. S., Nipping disease in the bud: nSMase2 inhibitors as therapeutics in extracellular vesicle-mediated diseases. Drug Discov Today 2021, 26 (7), 1656-1668.
Ibrahim, A.; Marban, E., Exosomes: Fundamental Biology and Roles in Cardiovascular Physiology. Annu Rev Physiol 2016, 78, 67-83.
Raposo, G.; Stoorvogel, W., Extracellular vesicles: exosomes, microvesicles, and friends. J Cell Biol 2013, 200 (4), 373-83.
Weidle, U. H.; Birzele, F.; Kollmorgen, G.; Ruger, R., The Multiple Roles of Exosomes in Metastasis. Cancer Genomics Proteomics 2017, 14 (1), 1-15.
Luberto, C.; Hassler, D. F.; Signorelli, P.; Okamoto, Y.; Sawai, H.; Boros, E.; Hazen-Martin, D. J.; Obeid, L. M.; Hannun, Y. A.; Smith, G. K., Inhibition of tumor necrosis factor-induced cell death in MCF7 by a novel inhibitor of neutral sphingomyelinase. J Biol Chem 2002, 277 (43), 41128-39.
Trajkovic, K.; Hsu, C.; Chiantia, S.; Rajendran, L.; Wenzel, D.; Wieland, F.; Schwille, P.; Brugger, B.; Simons, M., Ceramide triggers budding of exosome vesicles into multivesicular endosomes. Science 2008, 319 (5867), 1244-7.
Dickens, A. M.; Tovar, Y. R. L. B.; Yoo, S. W.; Trout, A. L.; Bae, M.; Kanmogne, M.; Megra, B.; Williams, D. W.; Witwer, K. W.; Gacias, M.; Tabatadze, N.; Cole, R. N.; Casaccia, P.; Berman, J. W.; Anthony, D. C.; Haughey, N. J., Astrocyte-shed extracellular vesicles regulate the peripheral leukocyte response to inflammatory brain lesions. Sci Signal 2017, 10 (473).
Kosaka, N.; Iguchi, H.; Hagiwara, K.; Yoshioka, Y.; Takeshita, F.; Ochiya, T., Neutral sphingomyelinase 2 (nSMase2)-dependent exosomal transfer of angiogenic microRNAs regulate cancer cell metastasis. J Biol Chem 2013, 288 (15), 10849-59.
Dinkins, M. B.; Enasko, J.; Hernandez, C.; Wang, G.; Kong, J.; Helwa, I.; Liu, Y.; Terry, A. V., Jr.; Bieberich, E., Neutral Sphingomyelinase-2 Deficiency Ameliorates Alzheimer's Disease Pathology and Improves Cognition in the 5XFAD Mouse. J Neurosci 2016, 36 (33), 8653-67.
Dinkins, M. B.; Dasgupta, S.; Wang, G.; Zhu, G.; Bieberich, E., Exosome reduction in vivo is associated with lower amyloid plaque load in the 5XFAD mouse model of Alzheimer's disease. Neurobiol Aging 2014, 35 (8), 1792-800.
Sardar Sinha, M.; Ansell-Schultz, A.; Civitelli, L.; Hildesjo, C.; Larsson, M.; Lannfelt, L.; Ingelsson, M.; Hallbeck, M., Alzheimer's disease pathology propagation by exosomes containing toxic amyloid-beta oligomers. Acta Neuropathol 2018, 136 (1), 41-56.
Asai, H.; Ikezu, S.; Tsunoda, S.; Medalla, M.; Luebke, J.; Haydar, T.; Wolozin, B.; Butovsky, O.; Kugler, S.; Ikezu, T., Depletion of microglia and inhibition of exosome synthesis halt tau propagation. Nat Neurosci 2015, 18 (11), 1584-93.
Barclay, R. A.; Schwab, A.; DeMarino, C.; Akpamagbo, Y.; Lepene, B.; Kassaye, S.; Iordanskiy, S.; Kashanchi, F., Exosomes from uninfected cells activate transcription of latent HIV-1. J Biol Chem 2017, 292 (28), 11682-11701.
Dalvi, P.; Sun, B.; Tang, N.; Pulliam, L., Immune activated monocyte exosomes alter microRNAs in brain endothelial cells and initiate an inflammatory response through the TLR4/MyD88 pathway. Sci Rep 2017, 7 (1), 9954.
Shukla, K.; Ferraris, D. V.; Thomas, A. G.; Stathis, M.; Duvall, B.; Delahanty, G.; Alt, J.; Rais, R.; Rojas, C.; Gao, P.; Xiang, Y.; Dang, C. V.; Slusher, B. S.; Tsukamoto, T., Design, Synthesis, and Pharmacological Evaluation of Bis-2-(5-phenylacetamido-1,2,4-thiadiazol-2-yl)ethyl Sulfide 3 (BPTES) Analogs as Glutaminase Inhibitors. J. Med. Chem. 2012, 55 (23), 10551-63.
Sun, B.; Dalvi, P.; Abadjian, L.; Tang, N.; Pulliam, L., Blood neuron-derived exosomes as biomarkers of cognitive impairment in HIV. AIDS 2017, 31 (14), F9-F17.
Rojas, C.; Barnaeva, E.; Thomas, A. G.; Hu, X.; Southall, N.; Marugan, J.; Chaudhuri, A. D.; Yoo, S.- W.; Hin, N.; Stepanek, O.; Wu, Y.; Zimmermann, S. C.; Gadiano, A. G.; Tsukamoto, T.; Rais, R.; Haughey, N.; Ferrer, M.; Slusher, B. S., DPTIP, a newly identified potent brain penetrant neutral sphingomyelinase 2 inhibitor, regulates astrocyte-peripheral immune communication following brain inflammation. Scientific Reports 2018, 8 (1), 17715.
Stepanek, O.; Hin, N.; Thomas, A. G.; Dash, R. P.; Alt, J.; Rais, R.; Rojas, C.; Slusher, B. S.; Tsukamoto, T., Neutral sphingomyelinase 2 inhibitors based on the 4-(1H-imidazol-2-yl)-2,6-dialkoxyphenol scaffold. European Journal of Medicinal Chemistry 2019, 170, 276-289.
Figuera-Losada, M.; Stathis, M.; Dorskind, J. M.; Thomas, A. G.; Bandaru, V. V.; Yoo, S. W.; Westwood, N. J.; Rogers, G. W.; McArthur, J. C.; Haughey, N. J.; Slusher, B. S.; Rojas, C., Cambinol, a novel inhibitor of neutral sphingomyelinase 2 shows neuroprotective properties. PLOS One 2015, 10 (5), e0124481.
Rojas, C.; Sala, M.; Thomas, A. G.; Datta Chaudhuri, A.; Yoo, S.- W.; Li, Z.; Dash, R. P.; Rais, R.; Haughey, N. J.; Nencka, R.; Slusher, B., A novel and potent brain penetrant inhibitor of extracellular vesicle release. British Journal of Pharmacology 2019, 176 (19), 3857-3870.
Aungst, B. J.; Hussain, M. A., Use of prodrugs of 3-hydroxymorphinans to prevent bitter taste upon buccal, nasal or sublingual administration. Google Patents: 1987.
Hussain, A. A.; AL-Bayatti, A. A.; Dakkuri, A.; Okochi, K.; Hussain, M. A., Testosterone 17β-N,N-dimethylglycinate hydrochloride: A prodrug with a potential for nasal delivery of testosterone. J. Pharm. Sci. 2002, 91 (3), 785-789.
Kao, H. D.; Traboulsi, A.; Itoh, S.; Dittert, L.; Hussain, A., Enhancement of the systemic and CNS specific delivery of L-dopa by the nasal administration of its water soluble prodrugs. Pharm. Res. 2000, 17 (8), 978-984.
Rautio, J.; Meanwell, N. A.; Di, L.; Hageman, M. J., The expanding role of prodrugs in contemporary drug design and development. Nat. Rev. Drug Discov. 2018.
Zimmermann, S. C.; Tichy, T.; Vavra, J.; Dash, R. P.; Slusher, C. E.; Gadiano, A. J.; Wu, Y.; Jancarik, A.; Tenora, L.; Monincova, L.; Prchalova, E.; Riggins, G. J.; Majer, P.; Slusher, B. S.; Rais, R., N-Substituted Prodrugs of Mebendazole Provide Improved Aqueous Solubility and Oral Bioavailability in Mice and Dogs. J Med Chem 2018, 61 (9), 3918-3929.
Dash, R. P.; Tichý, T.; Veeravalli, V.; Lam, J.; Alt, J.; Wu, Y.; Tenora, L.; Majer, P.; Slusher, B. S.; Rais, R., Enhanced Oral Bioavailability of 2-(Phosphonomethyl)-pentanedioic Acid (2-PMPA) from its (5-Methyl-2-oxo-1,3-dioxol-4-yl)methyl (ODOL)-Based Prodrugs. Molecular Pharmaceutics 2019, 16 (10), 4292-4301.
Rais, R.; Jancarik, A.; Tenora, L.; Nedelcovych, M.; Alt, J.; Englert, J.; Rojas, C.; Le, A.; Elgogary, A.; Tan, J.; Monincova, L.; Pate, K.; Adams, R.; Ferraris, D.; Powell, J.; Majer, P.; Slusher, B. S., Discovery of 6-Diazo-5-oxo-1-norleucine (DON) Prodrugs with Enhanced CSF Delivery in Monkeys: A Potential Treatment for Glioblastoma. J Med Chem 2016, 59 (18), 8621-33.
Tenora, L.; Alt, J.; Dash, R. P.; Gadiano, A. J.; Novotna, K.; Veeravalli, V.; Lam, J.; Kirkpatrick, Q. R.; Lemberg, K. M.; Majer, P.; Rais, R.; Slusher, B. S., Tumor-Targeted Delivery of 6-Diazo-5-oxo-1-norleucine (DON) Using Substituted Acetylated Lysine Prodrugs. J Med Chem 2019, 62 (7), 3524-3538.
Stepanek, O.; Hin, N.; Thomas, A. G.; Dash, R. P.; Alt, J.; Rais, R.; Rojas, C.; Slusher, B. S.; Tsukamoto, T., Neutral sphingomyelinase 2 inhibitors based on the 4-(1H-imidazol-2-yl)-2,6-dialkoxyphenol scaffold. Eur J Med Chem 2019, 170, 276-289.
Slatter, J. G.; Schaaf, L. J.; Sams, J. P.; Feenstra, K. L.; Johnson, M. G.; Bombardt, P. A.; Cathcart, K. S.; Verburg, M. T.; Pearson, L. K.; Compton, L. D.; Miller, L. L.; Baker, D. S.; Pesheck, C. V.; Lord, R. S., 3rd, Pharmacokinetics, metabolism, and excretion of irinotecan (CPT-11) following I.V. infusion of [(14)C]CPT-11 in cancer patients. Drug Metab Dispos 2000, 28 (4), 423-33.
Heimbach, T.; Oh, D. M.; Li, L. Y.; Forsberg, M.; Savolainen, J.; Leppänen, J.; Matsunaga, Y.; Flynn, G.; Fleisher, D., Absorption rate limit considerations for oral phosphate prodrugs. Pharm Res 2003, 20 (6), 848-56.
Zimmermann, S. C.; Tichý, T.; Vávra, J.; Dash, R. P.; Slusher, C. E.; Gadiano, A. J.; Wu, Y.; Jančařík, A.; Tenora, L.; Monincová, L.; Prchalová, E.; Riggins, G. J.; Majer, P.; Slusher, B. S.; Rais, R., N-Substituted Prodrugs of Mebendazole Provide Improved Aqueous Solubility and Oral Bioavailability in Mice and Dogs. Journal of Medicinal Chemistry 2018, 61 (9), 3918-3929.
Tenora, L.; Alt, J.; Dash, R. P.; Gadiano, A. J.; Novotná, K.; Veeravalli, V.; Lam, J.; Kirkpatrick, Q. R.; Lemberg, K. M.; Majer, P.; Rais, R.; Slusher, B. S., Tumor-Targeted Delivery of 6-Diazo-5-oxo-1-norleucine (DON) Using Substituted Acetylated Lysine Prodrugs. Journal of medicinal chemistry 2019, 62 (7), 3524-3538.
Rojas, C.; Barnaeva, E.; Thomas, A. G.; Hu, X.; Southall, N.; Marugan, J.; Chaudhuri, A. D.; Yoo, S. W.; Hin, N.; Stepanek, O.; Wu, Y.; Zimmermann, S. C.; Gadiano, A. G.; Tsukamoto, T.; Rais, R.; Haughey, N.; Ferrer, M.; Slusher, B. S., DPTIP, a newly identified potent brain penetrant neutral sphingomyelinase 2 inhibitor, regulates astrocyte-peripheral immune communication following brain inflammation. Sci Rep 2018, 8 (1), 17715.
Mccluskey, L.; Campbell, S.; Anthony, D.; Allan, S. M., Inflammatory responses in the rat brain in response to different methods of intra-cerebral administration. J Neuroimmunol 2008, 194 (1-2), 27-33.
Zhang, Q. G.; Wang, R.; Tang, H.; Dong, Y.; Chan, A.; Sareddy, G. R.; Vadlamudi, R. K.; Brann, D. W., Brain-derived estrogen exerts anti-inflammatory and neuroprotective actions in the rat hippocampus. Mol Cell Endocrinol 2014, 389 (1-2), 84-91.
Paxinos, G., and Franklin, K. B. J., The mouse brain in stereotaxic coordinates. Second ed.; Academic Press: 2001.
Hu G, Yao H, Chaudhuri A D, Duan M, Yelamanchili S V, Wen H, Cheney P D, Fox H S, Buch S. Exosome-mediated shuttling of microRNA-29 regulates HIV Tat and morphine-mediated neuronal dysfunction. Cell Death Dis. 2012;3:e381.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A compound of formula (I): 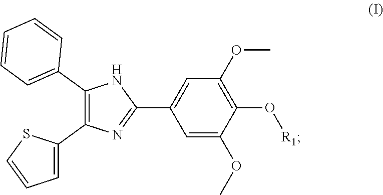
wherein:
R₁ is —C(═O)—R₂ or —(CH₂)ₙ—O—P(═O)(OH)(OR₃), wherein:
n is an integer selected from the group consisting of 0, 1, 2, 3, and 4;
R₂ is selected from the group consisting of substituted or unsubstituted C₁-C₈ straight-chain or branched alkyl, —NR₄R₅, substituted or unsubstituted cycloalkyl or cycloheteroalkyl, substituted or unsubstituted aryl or heteroaryl, substituted or unsubstituted bicycloalkyl or bicycloheteroalkyl, —O—CH(R₆)—O—(C═O)—R₇, and —CH(R₈)(NR₉)—(C═O)—CH(NR₁₀R₁₁)—R₁₂;
wherein:
R₄, R₅, R₈, R₉, R₁₀, and R₁₁ are each independently selected from the group consisting of H and substituted or unsubstituted straight-chain or branched C₁-C₈ alkyl;
R₆ is selected from the group consisting of substituted or unsubstituted straight-chain or branched C₁-C₄ alkyl and substituted or unsubstituted aryl or heteroaryl; and
R₇ and R₁₂ are each independently substituted or unsubstituted straight-chain or branched C₁-C₄ alkyl,
R₃ is H or —(CH₂)ₘ—O—C(═O)—O—R₁₃, wherein m is an integer selected from the group consisting of 1, 2, 3, and 4, and R₁₃ is substituted or unsubstituted C₁-C₄ alkyl;
and pharmaceutically acceptable salts thereof.

2. The compound of claim 1, wherein R₁ is —C(═O)—R₂.

3. The compound of claim 2, wherein R₂ is substituted or unsubstituted C₁-C₈ straight-chain or branched alkyl.

4. The compound of claim 3, wherein R₂ is selected from the group consisting of methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, and n-octyl.

5. The compound of claim 1, wherein R₂ is —NR₄R₅.

6. The compound of claim 1, wherein R₂ is selected from the group consisting of: 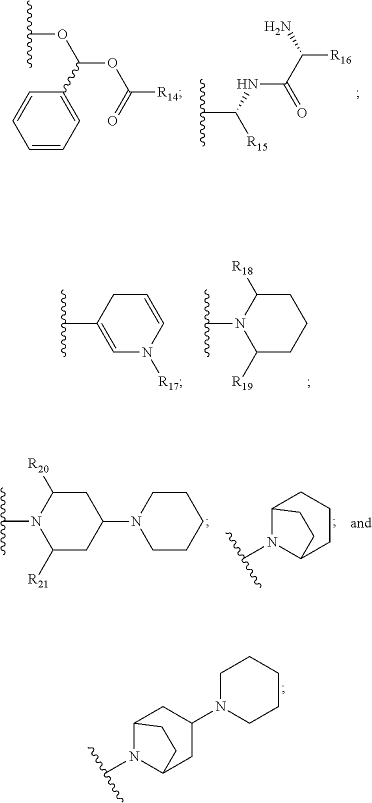
wherein R₁₄, R₁₅, R₁₆, R₁₈, and R₁₉ are each independently selected from the group consisting of substituted or unsubstituted straight-chain or branched alkyl, and R₁₇, R₂₀, and R₂₁ are each independently selected from the group consisting of H or C₁-C₄ substituted or unsubstituted C₁-C₄ straight-chain or branched alkyl.

7. The compound of claim 1, wherein R₁ is —(CH₂)ₙ—O—P(═O)(OH)(OR₃).

8. The compound of claim 7, wherein n is 0 or 1.

9. The compound of claim 7, wherein R₃ is H or —CH₂—O—C(═O)—O—R₁₃, wherein R₁₃ is selected from the group consisting of methyl, ethyl, n-propyl, isopropyl, n-butyl, isobutyl, sec-butyl, and tert-butyl.

10. The compound of claim 1, wherein the compound of formula (I) is selected from the group consisting of:

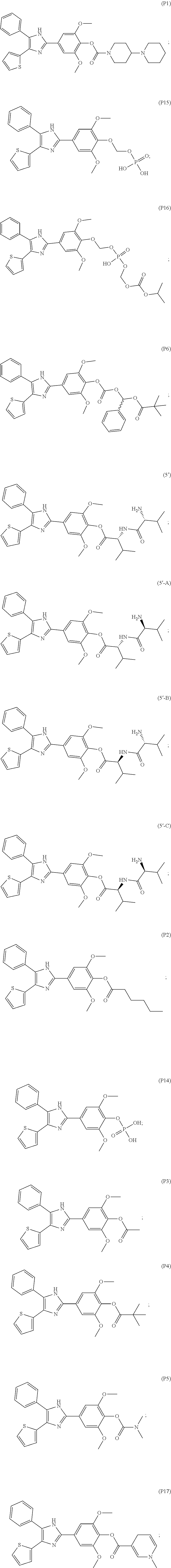

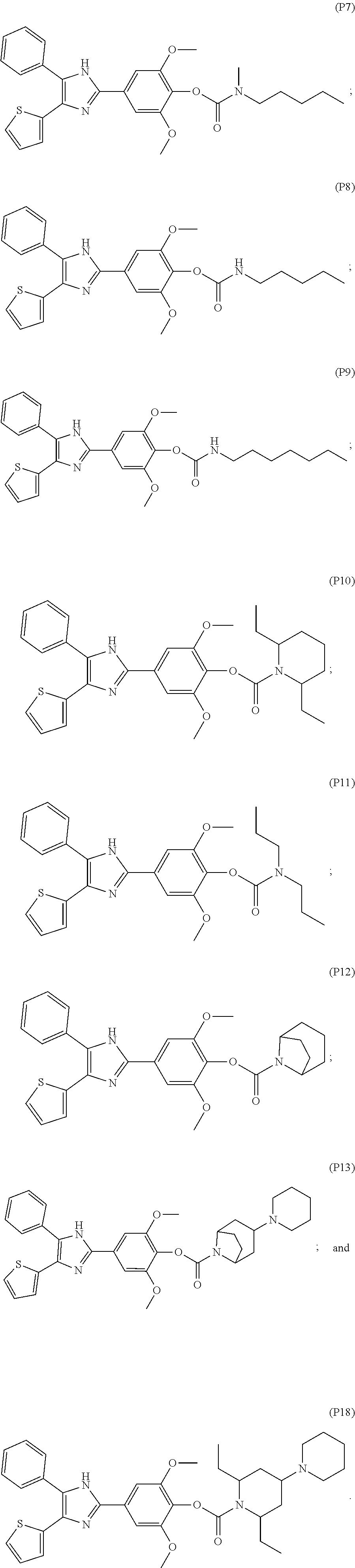

11. A pharmaceutical formulation comprising a compound of claim 1 and a pharmaceutically acceptable carrier.

12. The pharmaceutical formulation of claim 11, comprising one or more of a polyvinyl caprolactam-polyvinyl acetate-polyethylene glycol graft copolymer (PCL-PVAc-PEG), a caprylocaproyl macrogol-8 glyceride, and a cyclodextrin.

13. A method for treating a condition, disease, or disorder associated with an increased neutral sphingomyelinase 2 (nSMase2) activity or expression, the method comprising administering to a subject in need of treatment thereof an effective amount of a compound of claim 1, and pharmaceutically acceptable salts and pharmaceutical formulations thereof.

14. The method of claim 13, wherein the condition, disease, or disorder is associated with an elevated level of ceramide in the subject in need of treatment compared to a control subject not afflicted with the condition, disease, or disorder.

15. The method of claim 13, wherein the administration of an effective amount of a compound of claim 1 to the subject decreases the (nSMase2) activity or expression or decreases a level of ceramide in the subject.

16. The method of claim 13, wherein the condition, disease, or disorder is associated with an extracellular vesicle-mediated condition, disease, or disorder.

17. The method of claim 16, wherein the extracellular vesicle-mediated disease is selected from the group consisting of a neurological, an oncological, an inflammatory, and an infectious condition, disease, or disorder.

18. The method of claim 17, wherein the neurological condition, disease, or disorder is selected from the group consisting of Alzheimer's disease (AD), Parkinson's disease, HIV-associated neurocognitive disorder (HAND), multiple sclerosis (MS), amyotrophic lateral sclerosis (ALS), frontotemporal dementia (FTD), prion diseases, Duchenne muscular dystrophy (DMD), and retinal degeneration.

19. The method of claim 17, wherein the oncological condition, disease, or disorder is selected from the group consisting of breast cancer, cervical cancer, colon cancer, colorectal cancer (CRC), duodenal cancer, gastric cancer, lung cancer, multiple myeloma, oral cancer, pancreatic cancer, prostate cancer, skin cancer, and a cancer therapy in combination with immunotherapy to enhance systemic antitumor immunity.

20. The method of claim 17, wherein the inflammatory condition, disease, or disorder is selected from the group consisting of an inflammatory airway disease, including an allergic airway inflammation, an ischemia-reperfusion injury, including cerebral ischemia and hepatic ischemia-reperfusion injury, sepsis, atherosclerosis, myocardial infarction, and inflammatory bowel disease.

21. The method of claim 17, wherein the infectious condition, disease, or disorder comprises a viral infection selected from the group consisting of HIV, Zika virus, rabies virus, Dengue virus, hepatitis C (HCV), hepatitis E (HEV), cytomegalovirus (HCMV), Newcastle disease virus (NDV), and Langat virus.

22. The method of claim 17, wherein the infectious disease is related to a toxin produced from a bacterial infection, including Shiga toxin released by Escherichia coli, and epsilon toxin, released by Clostridum perfringens.

23. A method for inhibiting neutral sphingomyelinase 2 (nSMase2), the method comprising administering to a subject, cell, or tissue an amount of a compound of claim 1 effective to inhibit nSMase2.

24. Use of a compound of claim 1 for preparing a medicament for treating a condition, disease, or disorder associated with an increased neutral sphingomyelinase 2 (nSMase2) activity or expression.
